(19) United States
(12) Patent Application Publication
(10) Pub. No.: US 2025/0164396 A1
(43) Pub. Date: May 22, 2025

(54) METHOD FOR VISUALIZING DENATURED STATE OR AGGREGATED STATE OF PROTEIN

(71) Applicants: TOHOKU UNIVERSITY, Sendai-shi, Miyagi (JP); RIKEN, Wako-shi, Saitama (JP)

(72) Inventors: Shinichi SATO, Sendai-shi (JP); Keita NAKANE, Sendai-shi (JP); Kosuke DODO, Wako-shi (JP)

(73) Assignees: TOHOKU UNIVERSITY, Sendai-shi, Miyagi (JP); RIKEN, Wako-shi, Saitama (JP)

(21) Appl. No.: 18/729,763

(22) PCT Filed: Jan. 17, 2023
(86) PCT No.: PCT/JP2023/001094

§ 371 Date: Jul. 17, 2024

(30) Foreign Application Priority Data
Jan. 18, 2022 (JP) 2022-005871

(51) Int. Cl. G01N 21/64; C07D 271/12; G01N 21/77; G01N 33/68

(52) U.S. Cl. CPC G01N 21/64; C07D 271/12; G01N 21/77; G01N 33/68; G01N 2021/7786

(57) ABSTRACT

Provided is a method for visualizing the denatured state or aggregated state of proteins as a means for detecting denatured or aggregated proteins with high sensitivity, which can be applied even to a protein mixed system, the method comprising a steps of bringing a protein into contact with a nitrobenzoxadiazole derivative, and a steps of detecting the fluorescence of a reaction product of the nitrobenzoxadiazole derivative and the protein.

### TECHNICAL FIELD

The present invention relates to a method for visualizing the denatured state or aggregated state of proteins, a visualizing agent for the denatured state or aggregated state of proteins, a method for specifying denatured proteins or aggregated proteins in a sample, and a method for identifying proteins binding to a test compound.

### BACKGROUND ART

It is known that proteins can change to a denatured state or an aggregated state, such as amyloid fibers, by heating or under specific conditions. The former is an important phenomenon for basic research on proteins, and the latter is deeply involved in neurodegenerative diseases. Therefore, the development of a method for visualizing the denatured or aggregated state of proteins will have a great impact academically and industrially.

In conventional methods, low-molecular-weight chemical probes are widely used to visualize structures in which hydrophobic structures originally buried inside proteins are exposed to the protein surface during the heat denaturation process (denatured structures) and aggregated structures of beta-sheets etc. (Non-Patent Literature 1). Conventional fluorescent chemical probes are generally characterized by non-covalent interactions with denatured or aggregated structures to enhance fluorescence.

### CITATION LIST

### Non-Patent Literature

### [Non-Patent Literature 1]

Valtonen, Salla, Emmiliisa Vuorinen, Ville Eskonen, Morteza Malakoutikhah, Kari Kopra, and Harri Harma. 2021. “Sensitive, Homogeneous, and Label-Free Protein-Probe Assay for Antibody Aggregation and Thermal Stability Studies.” MAbs 13 (1): 1955810.

### SUMMARY OF INVENTION

### Technical Problem

When a conventional fluorescent probe is applied to a protein mixed system, it is difficult to observe which proteins have enhanced fluorescence in response to the denaturation or aggregation of proteins. Accordingly, the application is mainly used to observe the denaturation or aggregation of purified proteins.

Further, there is a demand for the development of fluorescent chemical probes that give high S/N ratios with higher sensitivity.

The present invention has been made under such a background, and an object thereof is to provide a means for detecting denatured or aggregated proteins with high sensitivity, which can be applied even to a protein mixed system.

### Solution to Problem

As a result of extensive research to achieve the above object, the present inventors have found that nitrobenzoxadiazole (NBD) selectively binds to denatured or aggregated proteins to emit fluorescence, and thus have completed the present invention.

Specifically, the present invention provides the following [1] to [17].
[1]A method for visualizing the denatured state or aggregated state of a protein, the method comprising: a step of bringing the protein into contact with a compound represented by the following general formula (I):

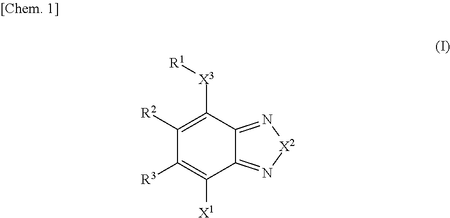

[wherein R¹ represents a C₁₋₁₂ linear alkyl group (provided that one or more —CH₂—CH₂— in the alkyl group are optionally replaced with —CO—NH—), a C₃₋₁₀ branched alkyl group (provided that one or more non-adjacent carbon atoms in the alkyl group are optionally replaced with a nitrogen atom or an oxygen atom), a C₃₋₁₀ cycloalkyl group optionally substituted with a substituent (provided that one or more non-adjacent carbon atoms in the cycloalkyl group are optionally replaced with a nitrogen atom or an oxygen atom), an aryl group optionally substituted with a substituent, a heteroaryl group optionally substituted with a substituent, a group represented by —(CH₂—CH₂—O—)ₙ—CH₃ (n represents an integer of 1 to 4), an adamantyl group, or a halogen atom; R² and R³ each independently represent a hydrogen atom or a C₁₋₃ linear or branched alkyl group; X¹ represents a nitro group or a group represented by —SO₂NR⁴R⁵(wherein R⁴ and R⁵ each independently represent a C₁₋₃ linear or branched alkyl group); X² represents a group represented by any of the following formulas (i) to (v):

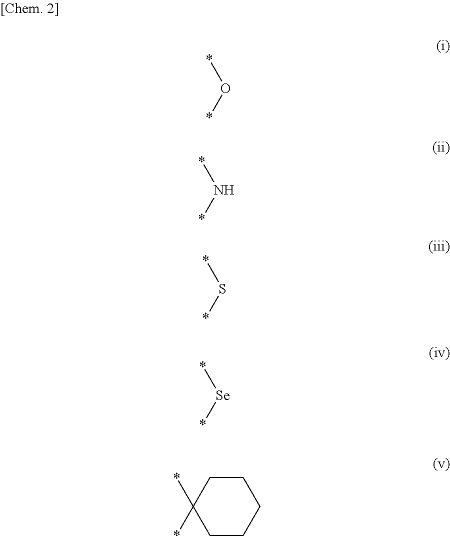

(wherein * represents a bond and binds to a nitrogen atom in the general formula (I)); and X³ does not exist or represents an oxygen atom or a sulfur atom]; and
a step of detecting fluorescence of a reaction product of the compound represented by the general formula (I).
[2] The method according to [1], wherein R¹ in the general formula (I) is a C₄₋₁₂ linear alkyl group (provided that one or more —CH₂—CH₂— in the alkyl group are optionally replaced with —CO—NH—), a C₃₋₅ branched alkyl group (provided that one or more non-adjacent carbon atoms in the alkyl group are optionally replaced with a nitrogen atom or an oxygen atom), a C₄₋₆ cycloalkyl group optionally substituted with a substituent (provided that one or more non-adjacent carbon atoms in the cycloalkyl group are optionally replaced with a nitrogen atom or an oxygen atom), a phenyl group optionally substituted with a substituent, a 2-thienyl group optionally substituted with a substituent, a 3-thienyl group optionally substituted with a substituent, a 2-furanyl group optionally substituted with a substituent, a 3-furanyl group optionally substituted with a substituent, a 2-pyridyl group optionally substituted with a substituent, a 3-pyridyl group optionally substituted with a substituent, a 4-pyridyl group optionally substituted with a substituent, or a group represented by —(CH₂—CH₂—O—)ₙ—CH₃ (n represents 2 or 3).
[3] The method according to [1], wherein R¹ in the general formula (I) is an n-butyl group, an n-octyl group, an iso-propyl group, a sec-butyl group, a tert-butyl group, a 3-pentyl group, a 1-methyl-2-methylamino-ethyl group, a cycloalkyl group, a 3-azetidyl group, a 3-piperidinyl group, a 3-aminomethylcyclobutyl group, a 3-aminocyclopentyl group, a 4-aminocyclohexyl group, a phenyl group, a 2-thienyl group, a 3-thienyl group, a 2-furanyl group, a 3-furanyl group, a 2-pyridyl group, a 3-pyridyl group, a 4-pyridyl group, or a group represented by —(CH₂—CH₂—O—)₂—CH₃.
[4] The method according to [1], wherein R¹ in the general formula (I) is an n-butyl group, an n-octyl group, an iso-propyl group, a 3-pentyl group, or a group represented by —(CH₂—CH₂—O—)₂—CH₃.
[5]A visualizing agent for the denatured state or aggregated state of a protein, the visualizing agent comprising a compound represented by the following general formula (I):

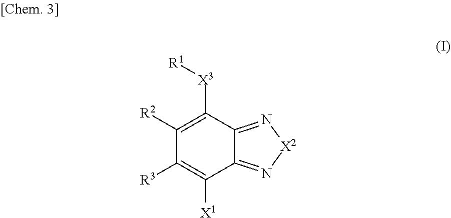

[wherein R¹ represents a C₁₋₁₂ linear alkyl group (provided that one or more —CH₂—CH₂— in the alkyl group are optionally replaced with —CO—NH—), a C₃₋₁₀ branched alkyl group (provided that one or more non-adjacent carbon atoms in the alkyl group are optionally replaced with a nitrogen atom or an oxygen atom), a C₃₋₁₀ cycloalkyl group optionally substituted with a substituent (provided that one or more non-adjacent carbon atoms in the cycloalkyl group are optionally replaced with a nitrogen atom or an oxygen atom), an aryl group optionally substituted with a substituent, a heteroaryl group optionally substituted with a substituent, a group represented by —(CH₂—CH₂—O—)ₙ—CH₃ (n represents an integer of 1 to 4), an adamantyl group, or a halogen atom; R² and R³ each independently represent a hydrogen atom or a C₁₋₃ linear or branched alkyl group; X¹ represents a nitro group or a group represented by —SO₂NR⁴R⁵(wherein R⁴ and R⁵ each independently represent a C₁₋₃ linear or branched alkyl group); X² represents a group represented by any of the following formulas (i) to (v):

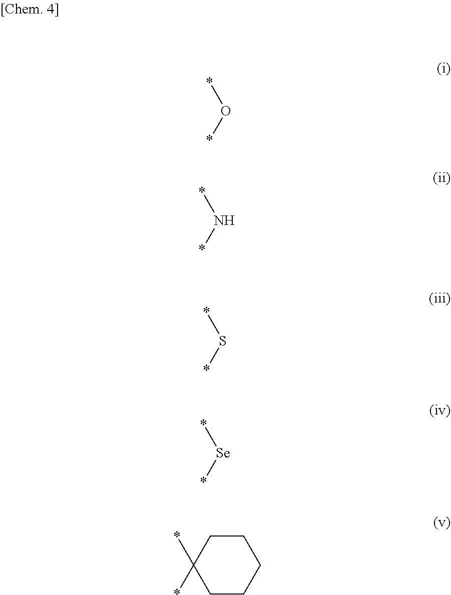

(wherein * represents a bond and binds to a nitrogen atom in the general formula (I)); and X³ does not exist or represents an oxygen atom or a sulfur atom].
[6] The visualizing agent according to [5], wherein R¹ in the general formula (I) is a C₄₋₁₂ linear alkyl group (provided that one or more —CH₂—CH₂— in the alkyl group are optionally replaced with —CO—NH—), a C₃₋₅ branched alkyl group (provided that one or more non-adjacent carbon atoms in the alkyl group are optionally replaced with a nitrogen atom or an oxygen atom), a C₄₋₆ cycloalkyl group optionally substituted with a substituent (provided that one or more non-adjacent carbon atoms in the cycloalkyl group are optionally replaced with a nitrogen atom or an oxygen atom), a phenyl group optionally substituted with a substituent, a 2-thienyl group optionally substituted with a substituent, a 3-thienyl group optionally substituted with a substituent, a 2-furanyl group optionally substituted with a substituent, a 3-furanyl group optionally substituted with a substituent, a 2-pyridyl group optionally substituted with a substituent, a 3-pyridyl group optionally substituted with a substituent, a 4-pyridyl group optionally substituted with a substituent, or a group represented by —(CH₂—CH₂—O—)ₙ—CH₃ (n represents 2 or 3).
[7] The visualizing agent according to [5], wherein R¹ in the general formula (I) is an n-butyl group, an n-octyl group, an iso-propyl group, a sec-butyl group, a tert-butyl group, a 3-pentyl group, a 1-methyl-2-methylamino-ethyl group, a cycloalkyl group, a 3-azetidyl group, a 3-piperidinyl group, a 3-aminomethylcyclobutyl group, a 3-aminocyclopentyl group, a 4-aminocyclohexyl group, a phenyl group, a 2-thienyl group, a 3-thienyl group, a 2-furanyl group, a 3-furanyl group, a 2-pyridyl group, a 3-pyridyl group, a 4-pyridyl group, or a group represented by —(CH₂—CH₂—O—)₂—CH₃.
[8] The visualizing agent according to [5], wherein R¹ in the general formula (I) is an n-butyl group, an n-octyl group, an iso-propyl group, a 3-pentyl group, or a group represented by —(CH₂—CH₂—O—)₂—CH₃.
[9]A method for specifying a denatured protein or an aggregated protein in a sample, the method comprising: a step of bringing the sample into contact with a compound represented by the following general formula (I):

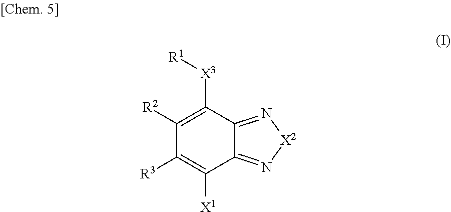

[wherein R¹ represents a C₁₋₁₂ linear alkyl group (provided that one or more —CH₂—CH₂— in the alkyl group are optionally replaced with —CO—NH—), a C₃₋₁₀ branched alkyl group (provided that one or more non-adjacent carbon atoms in the alkyl group are optionally replaced with a nitrogen atom or an oxygen atom), a C₃₋₁₀ cycloalkyl group optionally substituted with a substituent (provided that one or more non-adjacent carbon atoms in the cycloalkyl group are optionally replaced with a nitrogen atom or an oxygen atom), an aryl group optionally substituted with a substituent, a heteroaryl group optionally substituted with a substituent, a group represented by —(CH₂—CH₂—O—)ₙ—CH₃ (n represents an integer of 1 to 4), an adamantyl group, or a halogen atom; R² and R³ each independently represent a hydrogen atom or a C₁₋₃ linear or branched alkyl group; X¹ represents a nitro group or a group represented by —SO₂NR⁴R⁵(wherein R⁴ and R⁵ each independently represent a C₁₋₃ linear or branched alkyl group); X² represents a group represented by any of the following formulas (i) to (v):

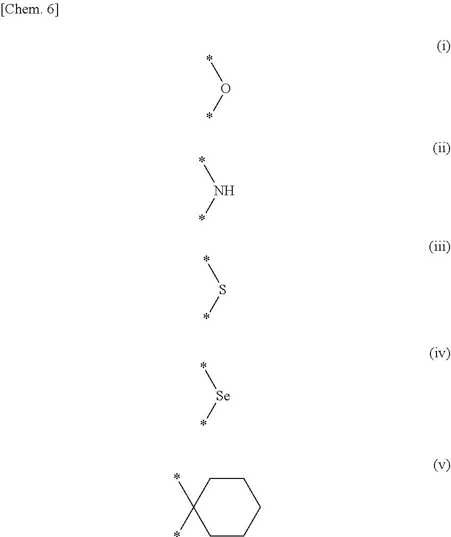

(wherein * represents a bond and binds to a nitrogen atom in the general formula (I)); and X³ does not exist or represents an oxygen atom or a sulfur atom];
a step of separating proteins in the sample; and
a step of detecting fluorescence of a reaction product of the compound represented by the general formula (I).
[10] The method according to [9], wherein R¹ in the general formula (I) is a C₄₋₁₂ linear alkyl group (provided that one or more —CH₂—CH₂— in the alkyl group are optionally replaced with —CO—NH—), a C₃₋₅ branched alkyl group (provided that one or more non-adjacent carbon atoms in the alkyl group are optionally replaced with a nitrogen atom or an oxygen atom), a C₄₋₆ cycloalkyl group optionally substituted with a substituent (provided that one or more non-adjacent carbon atoms in the cycloalkyl group are optionally replaced with a nitrogen atom or an oxygen atom), a phenyl group optionally substituted with a substituent, a 2-thienyl group optionally substituted with a substituent, a 3-thienyl group optionally substituted with a substituent, a 2-furanyl group optionally substituted with a substituent, a 3-furanyl group optionally substituted with a substituent, a 2-pyridyl group optionally substituted with a substituent, a 3-pyridyl group optionally substituted with a substituent, a 4-pyridyl group optionally substituted with a substituent, or a group represented by —(CH₂—CH₂—O—)ₙ—CH₃ (n represents 2 or 3).
[11] The method according to [9], wherein R¹ in the general formula (I) is an n-butyl group, an n-octyl group, an iso-propyl group, a sec-butyl group, a tert-butyl group, a 3-pentyl group, a 1-methyl-2-methylamino-ethyl group, a cycloalkyl group, a 3-azetidyl group, a 3-piperidinyl group, a 3-aminomethylcyclobutyl group, a 3-aminocyclopentyl group, a 4-aminocyclohexyl group, a phenyl group, a 2-thienyl group, a 3-thienyl group, a 2-furanyl group, a 3-furanyl group, a 2-pyridyl group, a 3-pyridyl group, a 4-pyridyl group, or a group represented by —(CH₂—CH₂—O—)₂—CH₃.
[12] The method according to [9], wherein R¹ in the general formula (I) is an n-butyl group, an n-octyl group, an iso-propyl group, a 3-pentyl group, or a group represented by —(CH₂—CH₂—O—)₂—CH₃.
[13]A method for identifying a protein binding to a test compound, the method comprising the following steps:
(1) adding the test compound and a fluorescence substance to a protein-containing sample;
(2) heating the sample of step (1);
(3) separating proteins contained in the sample of step (2);
(4) measuring fluorescence intensity of each protein separated in step (3);
(5) adding a fluorescence substance to a protein-containing sample;
(6) heating the sample of step (5);
(7) separating proteins contained in the sample of step (6);
(8) measuring fluorescence intensity of each protein separated in step (7); and
(9) comparing the fluorescence intensity of each protein measured in step (4) and the fluorescence intensity of each protein measured in step (8), and identifying a protein in which the fluorescence intensity measured in step (4) is lower than the fluorescence intensity measured in step (8) as a protein binding to the test compound, wherein the fluorescence substance is a compound represented by the following general formula (I):

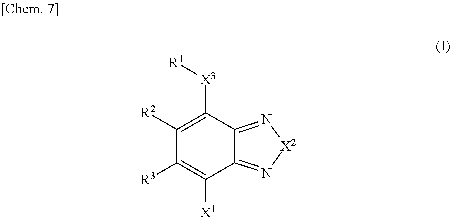

[wherein R¹ represents a C₁₋₁₂ linear alkyl group (provided that one or more —CH₂—CH₂— in the alkyl group are optionally replaced with —CO—NH—), a C₃₋₁₀ branched alkyl group (provided that one or more non-adjacent carbon atoms in the alkyl group are optionally replaced with a nitrogen atom or an oxygen atom), a C₃₋₁₀ cycloalkyl group optionally substituted with a substituent (provided that one or more non-adjacent carbon atoms in the cycloalkyl group are optionally replaced with a nitrogen atom or an oxygen atom), an aryl group optionally substituted with a substituent, a heteroaryl group optionally substituted with a substituent, a group represented by —(CH₂—CH₂—O—)ₙ—CH₃ (n represents an integer of 1 to 4), an adamantyl group, or a halogen atom; R² and R³ each independently represent a hydrogen atom or a C₁₋₃ linear or branched alkyl group; X¹ represents a nitro group or a group represented by —SO₂NR⁴R⁵(wherein R⁴ and R⁵ each independently represent a C₁₋₃ linear or branched alkyl group); X² represents a group represented by any of the following formulas (i) to (v):

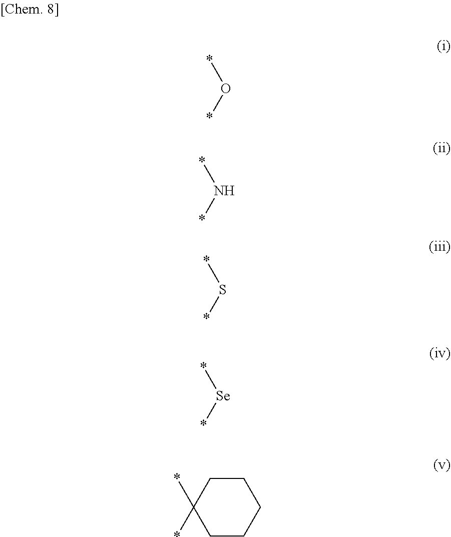

(wherein * represents a bond and binds to a nitrogen atom in the general formula (I)); and X³ does not exist or represents an oxygen atom or a sulfur atom].
[14] The method according to [13], wherein R¹ in the general formula (I) is a C₄₋₁₂ linear alkyl group (provided that one or more —CH₂—CH₂— in the alkyl group are optionally replaced with —CO—NH—), a C₃₋₅ branched alkyl group (provided that one or more non-adjacent carbon atoms in the alkyl group are optionally replaced with a nitrogen atom or an oxygen atom), a C₄₋₆ cycloalkyl group optionally substituted with a substituent (provided that one or more non-adjacent carbon atoms in the cycloalkyl group are optionally replaced with a nitrogen atom or an oxygen atom), a phenyl group optionally substituted with a substituent, a 2-thienyl group optionally substituted with a substituent, a 3-thienyl group optionally substituted with a substituent, a 2-furanyl group optionally substituted with a substituent, a 3-furanyl group optionally substituted with a substituent, a 2-pyridyl group optionally substituted with a substituent, a 3-pyridyl group optionally substituted with a substituent, a 4-pyridyl group optionally substituted with a substituent, or a group represented by —(CH₂—CH₂—O—)ₙ—CH₃ (n represents 2 or 3).
[15] The method according to [13], wherein R¹ in the general formula (I) is an n-butyl group, an n-octyl group, an iso-propyl group, a sec-butyl group, a tert-butyl group, a 3-pentyl group, a 1-methyl-2-methylamino-ethyl group, a cycloalkyl group, a 3-azetidyl group, a 3-piperidinyl group, a 3-aminomethylcyclobutyl group, a 3-aminocyclopentyl group, a 4-aminocyclohexyl group, a phenyl group, a 2-thienyl group, a 3-thienyl group, a 2-furanyl group, a 3-furanyl group, a 2-pyridyl group, a 3-pyridyl group, a 4-pyridyl group, or a group represented by —(CH₂—CH₂—O—)₂—CH₃.
[16] The method according to [13], wherein R¹ in the general formula (I) is an n-butyl group, an n-octyl group, an iso-propyl group, a 3-pentyl group, or a group represented by —(CH₂—CH₂—O—)₂—CH₃.
[17]A compound represented by the following general formula (Ia), (Ib), or (Ic):

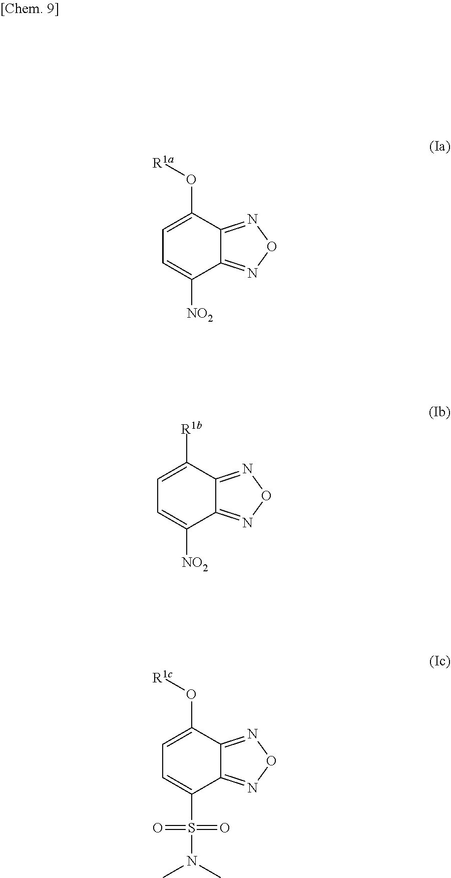

[wherein R^{1a} represents an iso-propyl group, an n-butyl group, a 3-pentyl group, a cyclobutyl group, a cyclopentyl group, a cyclohexyl group, a cycloheptyl group, a cyclooctyl group, a 2-adamantyl group, an n-hexyl group, an n-octyl group, an n-decyl group, an n-dodecyl group, an iso-butyl group, a 5-nonyl group, or a diethylene glycol group; R^{1b} represents a bromine atom or an iodine atom; and R^{1c} represents a methyl group or an iso-propyl group].

The present specification includes the contents described in the specification and/or drawings of Japanese Patent Application No. 2022-005871, which is the basis for the priority of this application.

### Advantageous Effects of Invention

The present invention provides a novel method for visualizing the denatured state or aggregated state of proteins. This method can visualize the denatured state or aggregated state of proteins with higher sensitivity than conventional methods using fluorescent chemical probes. Further, unlike conventional methods, fluorescent probes covalently bind to denatured or aggregated proteins, which remain as a history of denaturation or aggregation; thus, this method can be used in protein mixed systems and can be applied to proteomics analysis.

### BRIEF DESCRIPTION OF DRAWINGS

FIG. 1 shows the evaluation results of the improvement in fluorescence properties in response to heating.

FIG. 2 shows the quantification results of the aggregated state of amyloid β1-40.

FIG. 3 shows the quantification results of the aggregated state of α-synuclein.

FIG. 4 shows the detection results of heat-denatured proteins by two-dimensional electrophoresis (protocol to collect all proteins). The top row shows fluorescent images by N-NBD, the middle row shows a silver-stained image, and the bottom row shows magnified images of the fluorescent images of the upper row.

FIG. 5 shows the detection results of heat-denatured proteins by two-dimensional electrophoresis (protocol to collect pellets). The top row shows fluorescent images by N-NBD, the middle row shows Oriole-stained images, and the bottom row shows magnified images of the fluorescent images of the upper row.

FIG. 6 shows NBD derivatives and probe characteristics for the detection of heat denaturation of carbonic anhydrase.

FIG. 7 shows DBD derivatives and probe characteristics for the detection of heat denaturation of carbonic anhydrase.

FIG. 8 shows the reactivity of probes in cells.

FIG. 9 shows the results of staining denatured proteins with Octyl-ONBD in cells in the coexistence of a proteasome inhibitor.

FIG. 10 shows the results of staining denatured proteins with Octyl-ONBD in cells without or with addition of a proteasome inhibitor.

FIG. 11 shows the quantification results of the aggregated state of amyloid β1-40.

FIG. 12-1 shows the results of specification and quantification of probe reaction sites of carbonic anhydrase (CA).

FIG. 12-2 shows the results of specification and quantification of probe reaction sites of carbonic anhydrase (CA).

FIG. 12-3 shows the results of specification and quantification of probe reaction sites of carbonic anhydrase (CA).

FIG. 12-4 shows the results of specification and quantification of probe reaction sites of carbonic anhydrase (CA).

FIG. 13 shows the enrichment of labeled peptide fragments with anti-NBD antibody and the detection results by mass spectrometry.

FIG. 14 shows the visualization of the denatured state of CA by two-dimensional electrophoresis and the influence of the presence or absence of a ligand (when using BrNDB).

FIG. 15 shows the visualization of the denatured state of CA by two-dimensional electrophoresis and the influence of the presence or absence of a ligand (comparison between iPrONBD and BrNBD).

FIG. 16 shows the influence of the addition of geldanamycin on HSP90 precipitation and the results of fluorescence detection of denatured HSP90 by probes.

FIG. 17 shows the comparison of each probe in fluorescence detection of denatured HSP90 by probes by the addition of geldanamycin.

FIG. 18 shows the reaction of probe molecules in the intracellular environment (comparison of denatured HSP90 by the addition of geldanamycin).

FIG. 19 shows the results of spot-cutting from two-dimensional electrophoresis gel after denaturation detection and identification by mass spectrometry.

### DESCRIPTION OF EMBODIMENTS

The present invention will be described in detail below.

In the present invention, the “C₁₋₁₂ linear alkyl group” is, for example, a methyl group, an ethyl group, an n-propyl group, an n-butyl group, an n-pentyl group, an n-hexyl group, an n-heptyl group, an n-octyl group, an n-nonyl group, an n-decyl group, an n-undecyl group, or an n-dodecyl group.

In the present invention, the “C₄₋₁₂ linear alkyl group” is, for example, an n-butyl group, an n-pentyl group, an n-hexyl group, an n-heptyl group, an n-octyl group, an n-nonyl group, an n-decyl group, an n-undecyl group, or an n-dodecyl group.

In the present invention, the “C₃₋₁₀ (branched alkyl group” is, for example, an iso-propyl group, an iso-butyl group, a sec-butyl group, a tert-butyl group, an iso-pentyl group, a sec-pentyl group, a tert-pentyl group, a 3-pentyl group, a neopentyl group, an iso-hexyl group, a sec-hexyl group, a tert-hexyl group, a neohexyl group, an iso-heptyl group, a sec-heptyl group, a tert-heptyl group, a neoheptyl group, an iso-octyl group, a sec-octyl group, a tert-octyl group, a neooctyl group, an iso-nonyl group, a sec-nonyl group, a tert-nonyl group, a neononyl group, an iso-decyl group, a sec-decyl group, a tert-decyl group, or a neodecyl group.

In the present invention, the “C₃₋₅ branched alkyl group” is, for example, an iso-propyl group, an iso-butyl group, a sec-butyl group, a tert-butyl group, an iso-pentyl group, a sec-pentyl group, a tert-pentyl group, a 3-pentyl group, or a neopentyl group.

In the present invention, the “C₃₋₁₀ cycloalkyl group” is, for example, a cyclopropyl group, a cyclobutyl group, a cyclopentyl group, a cyclohexyl group, a cycloheptyl group, a cyclooctyl group, a cyclononyl group, or a cyclodecyl group.

In the present invention, the “C₄₋₆ cycloalkyl group” is, for example, a cyclobutyl group, a cyclopentyl group, or a cyclohexyl group.

In the present invention, the “aryl group” is, for example, a phenyl group, a 1-naphthyl group, or a 2-naphthyl group.

In the present invention, the “heteroaryl group” is, for example, a 2-thienyl group, a 3-thienyl group, a 2-furanyl group, a 3-furanyl group, a 2-pyridyl group, a 3-pyridyl group, or a 4-pyridyl group.

### (1) Visualization Method and Visualizing Agent

The method for visualizing the denatured state or aggregated state of proteins according to the present invention characteristically comprises a step of bringing a protein into contact with a compound represented by the following general formula (I), and a step of detecting fluorescence of a reaction product of the compound represented by the following general formula (I). The visualizing agent for the denatured state or aggregated state of proteins according to the present invention characteristically comprises the compound represented by the following general formula (I).

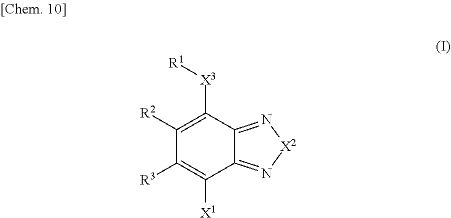

First, the principle of the method for visualizing the denatured state or aggregated state of proteins according to the present invention is explained. The compound represented by the general formula (I) does not emit fluorescence in a normal state; however, when this compound approaches a protein, it reacts with the amino group of the lysine residue of the protein (the carbon atom on the aromatic ring, to which X³ binds, and the nitrogen atom of the amino group of the lysine residue are covalently bonded) and begins to emit fluorescence (Yamaguchi, T. et al., Chem. Sci. 5, 1021-1029 (2014)). Since the compound represented by the general formula (I) is hydrophobic, whereas the surface of proteins that are not denatured or aggregated is hydrophilic, the compound represented by the general formula (I) hardly binds to the proteins that are not denatured or aggregated. On the other hand, when a protein is denatured or aggregated, a hydrophobic structure is formed on the surface of the protein; thus, the compound represented by the general formula (I) binds to this structure and emits fluorescence. Therefore, due to the fluorescence emitted by the reaction product of the compound represented by the general formula (I), the denatured state and aggregated state of proteins can be visualized.

In the general formula (I), R¹ may represent a linear alkyl group. The linear alkyl group is generally a C₁₋₁₂ linear alkyl group, preferably a C₄₋₁₂ linear alkyl group, more preferably an n-butyl group or an n-octyl group, and even more preferably an n-octyl group. When the linear alkyl group contains —CH₂—CH₂— (i.e., 2 or more carbon atoms, preferably 3 or more carbon atoms), —CH₂—CH₂— is optionally replaced with —CO—NH—. The number of such replacements in the linear alkyl group may be only one, or two or three.

In the general formula (I), R¹ may represent a branched alkyl group. The branched alkyl group is generally a C₃₋₁₀ branched alkyl group, preferably a C₃₋₅ branched alkyl group, more preferably an iso-propyl group, a sec-butyl group, a tert-butyl group, or a 3-pentyl group, and even more preferably an iso-propyl group. One or more non-adjacent carbon atoms in the branched alkyl group are optionally replaced with a nitrogen atom or an oxygen atom. The number of such replacements in the branched alkyl group may be only one, or two or three. Specific examples of branched alkyl groups whose carbon atom is replaced with a nitrogen atom or an oxygen atom include a 1-methyl-2-methylamino-ethyl group.

In the general formula (I), R¹ may represent a cycloalkyl group. The cycloalkyl group is generally a C₃₋₁₀ cycloalkyl group, preferably a C₄₋₆ cycloalkyl group, and more preferably a cyclohexyl group. One or more non-adjacent carbon atoms in the cycloalkyl group are optionally replaced with a nitrogen atom or an oxygen atom. The number of such replacements in the cycloalkyl group may be only one, or two or three. Specific examples of cycloalkyl groups whose carbon atom is replaced with a nitrogen atom or an oxygen atom include a 3-azetidyl group, a 3-tetrahydrofuranyl group, a 3-piperidinyl group, and a 4-tetrahydropyranyl group. The cycloalkyl group is optionally substituted with a substituent. Examples of substituents include an amino group and an aminomethyl group. Specific examples of cycloalkyl groups substituted with a substituent include a 3-aminomethylcyclobutyl group, a 3-aminocyclopentyl group, and a 4-aminocyclohexyl group.

In the general formula (I), R¹ may represent an aryl group. The aryl group is preferably a phenyl group. The aryl group may be substituted with a substituent. Examples of substituents include an amino group and an aminomethyl group.

In the general formula (I), R¹ may represent a heteroaryl group. The heteroaryl group is preferably 2-thienyl, 3-thienyl, 2-furanyl, 3-furanyl, 2-pyridyl, 3-pyridyl, or 4-pyridyl. The heteroaryl group may be substituted with a substituent. Examples of substituents include an amino group and an aminomethyl group.

In the general formula (I), R¹ may represent a group represented by (CH₂—CH₂—O—)ₙ—CH₃. Here, n is generally an integer of 1 to 4, and preferably 2 or 3.

In the general formula (I), R¹ may represent an adamantyl group. The adamantyl group may be any of a 1-adamantyl group and a 2-adamantyl group, but is preferably a 2-adamantyl group.

In the general formula (I), R¹ may represent a halogen atom. Examples of the halogen atom include a fluorine atom, a chlorine atom, a bromine atom, an iodine atom, and an astatine atom. Preferred among these are a bromine atom and an iodine atom.

In the general formula (I), R¹ may be a C₁₋₁₂ linear alkyl group (provided that one or more —CH₂—CH₂— in the alkyl group are optionally substituted with —CO—NH—), a C₃₋₁₀ branched alkyl group (provided that one or more non-adjacent carbon atoms in the alkyl group are optionally replaced with a nitrogen atom or an oxygen atom), a C₃₋₁₀ cycloalkyl group optionally substituted with a substituent (provided that one or more non-adjacent carbon atoms in the cycloalkyl group are optionally replaced with a nitrogen atom or an oxygen atom), an aryl group optionally substituted with a substituent, a heteroaryl group optionally substituted with a substituent, a group represented by —(CH₂—CH₂—O—)ₙ—CH₃ (n represents an integer of 1 to 4), an adamantyl group, or a halogen atom; preferably a C₄₋₁₂ linear alkyl group (provided that one or more —CH₂—CH₂— in the alkyl group are optionally replaced with —CO—NH—), a C₃₋₅ branched alkyl group (provided that one or more non-adjacent carbon atoms in the alkyl group are optionally replaced with a nitrogen atom or an oxygen atom), a C₄₋₆ cycloalkyl group optionally substituted with a substituent (provided that one or more non-adjacent carbon atoms in the cycloalkyl group are optionally replaced with a nitrogen atom or an oxygen atom), phenyl group optionally substituted with a substituent, a 2-thienyl group optionally substituted with a substituent, a 3-thienyl group optionally substituted with a substituent, a 2-furanyl group optionally substituted with a substituent, a 3-furanyl group optionally substituted with a substituent, a 2-pyridyl group optionally substituted with a substituent, a 3-pyridyl group optionally substituted with a substituent, a 4-pyridyl group optionally substituted with a substituent, or a group represented by —(CH₂—CH₂—O—)ₙ—CH₃ (n represents 2 or 3); more preferably an n-butyl group, an n-octyl group, an iso-propyl group, a sec-butyl group, a tert-butyl group, a 3-pentyl group, a 1-methyl-2-methylamino-ethyl group, a cycloalkyl group, a 3-azetidyl group, a 3-piperidinyl group, a 3-aminomethylcyclobutyl group, a 3-aminocyclopentyl group, a 4-aminocyclohexyl group, a phenyl group, a 2-thienyl group, a 3-thienyl group, a 2-furanyl group, a 3-furanyl group, a 2-pyridyl group, a 3-pyridyl group, a 4-pyridyl group, or a group represented by —(CH₂—CH₂—O—)₂—CH₃; and even more preferably an n-butyl group, an n-octyl group, an iso-propyl group, a 3-pentyl group, or a group represented by —(CH₂—CH₂—O—)₂—CH₃.

In the general formula (I), R² and R³ each independently represent a hydrogen atom or an alkyl group. The alkyl group is generally a C₁₋₃ linear or branched alkyl group, and preferably a methyl group. R² and R³ may be the same group or different groups. R² and R³ may be the groups mentioned above, but are preferably hydrogen atoms.

In the general formula (I), X¹ represents a nitro group or a group represented by —SO₂NR⁴R⁵. Here, R⁴ and R⁵ each independently represent an alkyl group. The alkyl group is generally a C₁₋₃ linear or branched alkyl group, and preferably a methyl group. R⁴ and R⁵ may be the same group or different groups. X¹ may be the group mentioned above, but is preferably a nitro group.

In the general formula (I), X² represents a group represented by any of the following formulas (i) to (v):

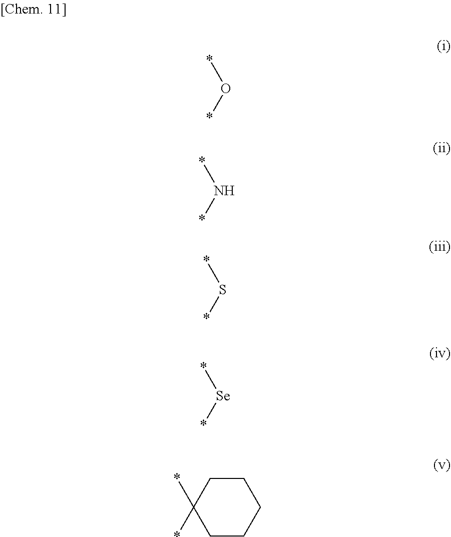

(wherein * represents a bond and binds to a nitrogen atom in the general formula (I)).

X² may be the group mentioned above, but is preferably the group represented by the formula (i).

In the general formula (I), X³ does not exist or represents an oxygen atom or a sulfur atom. X³ may be the group mentioned above, but is preferably an oxygen atom. However, when R¹ represents a halogen atom, X³ preferably does not exist. The phrase that “X³ does not exist” means that R¹ directly binds to the benzoxadiazole ring.

The proteins to be visualized are not particularly limited, and proteins that may be denatured and proteins that may be aggregated can be targeted for visualization. Denaturation mainly means heat denaturation, but may be denaturant denaturation, acid denaturation, pressure denaturation, and the like. Specific examples of proteins that may be aggregated include amyloid β, α-synuclein, and the like.

The method for bringing proteins into contact with the compound represented by the general formula (I) is not particularly limited. For example, a method that adds the compound represented by the general formula (I) into a protein-containing solution can be used.

The amount of the compound represented by the general formula (I) used is not particularly limited, but is generally 100 to 10000 mol, and preferably 100 to 1000 mol, per mol of the protein to be visualized.

The reaction product of the compound represented by the general formula (I) (protein-binding product) emits fluorescence. This fluorescence can be detected according to a conventional method. Of the compounds represented by the general formula (I), a nitrobenzoxadiazole derivative (N-NBD), wherein X³ contains a nitrogen atom, is known to emit fluorescence. Accordingly, the fluorescence of the reaction product of the compound represented by the general formula (I) can be detected by the same method.

In the present invention, “visualization of the denatured state or aggregated state of proteins” means that the denatured state or aggregated state of proteins is made visible. For example, clarifying whether the target proteins are denatured or aggregated, or clarifying how much the target proteins are denatured or aggregated (quantification of the degree of denaturation or aggregation) corresponds to the “visualization of the denatured state or aggregated state of proteins” in the present invention.

### (2) Specification Method

The method for specifying denatured proteins or aggregated proteins in a sample according to the present invention characteristically comprises a step of bringing the sample into contact with the compound represented by the general formula (I) described above, a step of separating proteins in the sample, and a step of detecting fluorescence of a reaction product of the compound represented by the general formula (I).

First, the principle of this method is explained. There are many known compounds that bind to denatured proteins or aggregated proteins (e.g., Thioflavin T binds to amyloid β, which is an aggregated protein). Since these compounds generally non-covalently bind to proteins, they are separated from the proteins when electrophoresis or the like is performed. Therefore, it is possible to use such compounds to clarify whether denatured proteins or aggregated proteins are present in the sample; however, if the sample contains a plurality of proteins, it is impossible to specify which proteins are denatured proteins or aggregated proteins. In contrast, the compound represented by the general formula (I) covalently binds to lysine residues of proteins, as described above, and is thus not easily separated from the proteins. Accordingly, even if the sample contains a plurality of proteins, it is possible to specify which proteins are denatured proteins or aggregated proteins by detecting the fluorescence of reaction products of the compound represented by the general formula (I) with proteins by a protein separation operation, such as electrophoresis.

As the sample, a sample containing only a single protein may be used; however, in general, a sample containing a plurality of proteins is used.

The method for bringing the sample into contact with the compound represented by the general formula (I) is not particularly limited. For example, a method that adds the compound represented by the general formula (I) into the sample can be used.

Proteins in the sample can be separated by a method commonly used for the separation of proteins, for example, electrophoresis or gel filtration.

The fluorescence of the reaction product of the compound represented by the general formula (I) can be detected in the same manner as in the visualization method of the present invention described above.

### (3) Identification Method

The method for identifying proteins binding to a test compound according to the present invention comprises the following steps (1) to (9).

In step (1), a test compound and the compound represented by the general formula (I) are added to a protein-containing sample. The protein-containing sample is not particularly limited, and is, for example, a cell disruption solution or a cell extract. The test compound is also not particularly limited, and is, for example, a bioactive compound.

In step (2), the sample of step (1) is heated. The heating conditions are not particularly limited as long as they are temperature and time at which proteins are denatured. The heating temperature can be, for example, 40 to 80° C., and the heating time can be, for example, 1 to 30 minutes.

In step (3), proteins contained in the sample of step (2) are separated. Proteins in the sample can be separated by a method commonly used for the separation of proteins, for example, electrophoresis or gel filtration.

In step (4), the fluorescence intensity of each protein separated in step (3) is measured. The fluorescence intensity can be measured according to a conventional method.

In step (5), the compound represented by the general formula (I) is added to a protein-containing sample. Step (5) can be performed in the same manner as in step (1), except that the test compound is not added.

In step (6), the sample of step (5) is heated. Step (6) can be performed in the same manner as in step (2).

In step (7), proteins contained in the sample of step (6) are separated. Step (7) can be performed in the same manner as in step (3).

In step (8), the fluorescence intensity of each protein separated in step (7) is measured. Step (8) can be performed in the same manner as in step (4).

In step (9), the fluorescence intensity of each protein measured in step (4) and the fluorescence intensity of each protein measured in step (8) are compared to identify proteins in which the fluorescence intensity measured in step (4) is lower than the fluorescence intensity measured in step (8) as proteins binding to the test compound. The principle that can identify proteins binding to the test compound by comparing the fluorescence intensities is as follows. When the test compound binds to a protein contained in the sample, the protein acquires heat denaturation resistance to reduce the degree of heat denaturation. The reduction in the degree of heat denaturation can be detected as a decrease in the fluorescence intensity of the compound represented by the general formula (I). Therefore, proteins in which the fluorescence intensity measured in step (4) is lower than the fluorescence intensity measured in step (8) are proteins that acquire heat denaturation resistance and bind to the test compound.

### EXAMPLES

The present invention will be described in more detail below with reference to Examples; however, the present invention is not limited to these Examples.

### [Example 1] Synthesis of compounds

### 1-1. iPrONBD

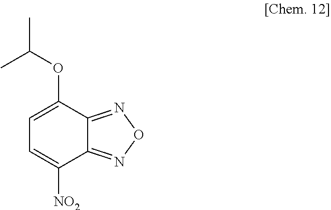 [SMILES: CC(C)Oc1ccc([N+](=O)[O-])c2nonc12]

F-NBD (TCI, A5593; 20.0 mg, 110 μmol) and propan-2-ol (TCI, 10277; 1 mL) were added to a ChemiStation (EYELA, Personal Organic Synthesizer) tube (capacity: 15 mL) and reacted at 60° C. for 23 hours. After completion of the reaction, the resultant was dried under reduced pressure and then separated by column chromatography (hexane: AcOEt), thereby obtaining iPrONBD as a green amorphous solid (11.3 mg, 46%, 50.6 μmol). ¹H NMR (600 MHz, CDCl₃) δ_{H} 8.54 (d, J=9.0 Hz, 1H), 6.66 (d, J=8.4 Hz, 1H), 5.05 (sept, J=6.0 Hz, 1H), 1.57 (d, J=6.0 Hz, 6H); ¹³C NMR (150 MHz, CDCl₃) δ_{C} 154.3, 145.8, 144.3, 134.4, 129.3, 105.0, 74.9, 21.7; HRMS (ESI, positive): calcd. for [C₉H₉N₃O₄+Na]⁺246.0485, found 246.0487.

### 1-2. nBuONBD

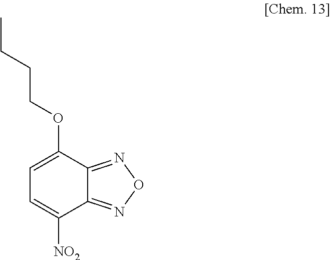 [SMILES: CCCCOc1ccc([N+](=O)[O-])c2nonc12]

F-NBD (25.0 mg, 140 μmol), butanol (TCI, B0944; 310 μL, 3.40 mmol), DIEPA (Nacalai, 14014-42; N-ethyl-N-isopropylpropan-2-amine, 138 μL, 140 μmol), and CH₂Cl₂ (Nacalai, 22430-74; 3.8 mL) were added to a ChemiStation (EYELA, Personal Organic Synthesizer) tube (capacity: 15 mL) and reacted at room temperature for 24 hours. After completion of the reaction, the resultant was dried under reduced pressure and then separated by column chromatography (hexane: AcOEt), thereby obtaining nBuONBD as a yellow solid (27.0 mg, 83%, 110 μmol). ¹H NMR (600 MHz, CDCl₃) δ_{H} 8.54 (d, J=8.4 Hz, 1H), 6.68 (d, J=9.0 Hz, 1H), 4.40 (t, J=6.6 Hz, 1H), 2.00-1.96 (m, 2H), 1.62-1.56 (m, 2H), 1.02 (t, J=7.2 Hz, 3H); ¹³C NMR (150 MHz, CDCl₃) δ_{C} 155.2, 145.4, 144.1, 134.3, 129.6, 104.4, 71.3, 30.7, 19.2, 13.8; HRMS (ESI, positive): calcd. for [C₁₀H₁₂N₃O₄+Na]⁺260.0642, found 260.0645.

### 1-3. 3-PentylONBD

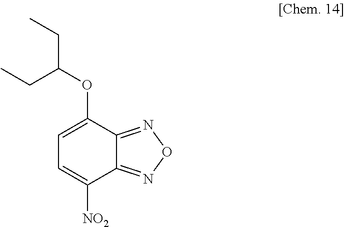 [SMILES: CCC(CC)Oc1ccc([N+](=O)[O-])c2nonc12]

F-NBD (38.4 mg, 210 μmol) and pentan-3-ol (Nacalai, 02717-12; 1 mL) were added to a ChemiStation (EYELA, Personal Organic Synthesizer) tube (capacity: 15 mL) and reacted at 60° C. for 22 hours. After completion of the reaction, the resultant was dried under reduced pressure and then separated by PTLC using CHCl₃, thereby obtaining 3-pentylONBD as a green amorphous solid (8.8 mg, 17%, 35 mol). ¹H NMR (600 MHz, CDCl₃) δ_{H} 8.54 (d, J=8.4 Hz, 1H), 6.64 (d, J=8.4 Hz, 1H), 4.67 (quint, J=6.0 Hz, 1H), 1.94-1.85 (m, 4H) 1.04 (t, J=7.2 Hz, 6H); ¹³C NMR (150 MHz, CDCl₃) δ_{C} 155.1, 145.8, 144.3, 134.4, 129.2, 105.1, 84.9, 26.1, 9.6; HRMS (ESI, negative): calcd. for [C₁₁H₁₃N₃O₄H]⁻ 250.0822, found 250.0856.

### [Example 2] Evaluation of improvement in fluorescence properties in response to heating

Bovine carbonic anhydrase (Aldrich, C3934) was diluted with 10 mM PBS to a final concentration of 10 M. A 1 mM DMSO solution of brinzolamide (TCI, B4258) was added to 1 equivalent of protein, and the mixture was allowed to stand at room temperature for 30 minutes. For the comparison target, the same amount of DMSO was added, and the same operation was carried out. Subsequently, a stock solution (DMSO solution) of 100 mM ONBD compound was added to a final concentration of 1 mM, and after gentle stirring with a vortex, 50 μL of the solution was transferred to a PCR tube. The PCR tube was heated with a protocol to increase the temperature by 5° C. every 180 seconds from 25° C. using a real-time PCR system (Takara Bio Inc., Thermal Cycler Dice, TP800). The fluorescence (FAM mode for ONBD compounds) was measured at the last 15 seconds of each step. Sypro Orange (Invitrogen S6650, 5000× concentration in DMSO) of the comparison target was added to a final concentration of 10×, the same operation was carried out, and the fluorescence was measured in ROX mode. The ONBD compounds used were the following five types: MeONBD, iPrONBD, nBuONBD, iBuONBD, and octylONBD.

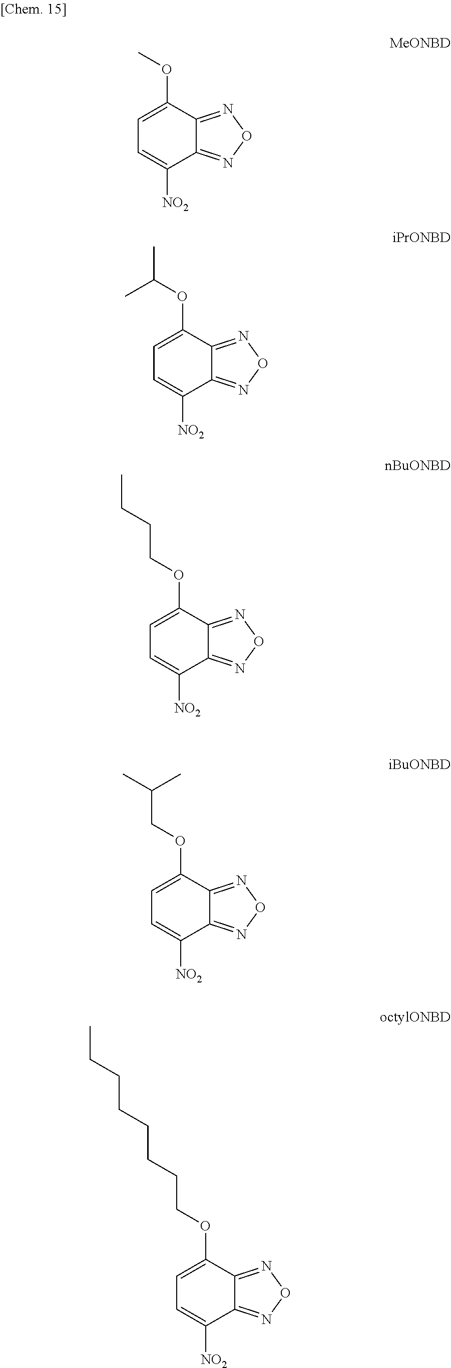

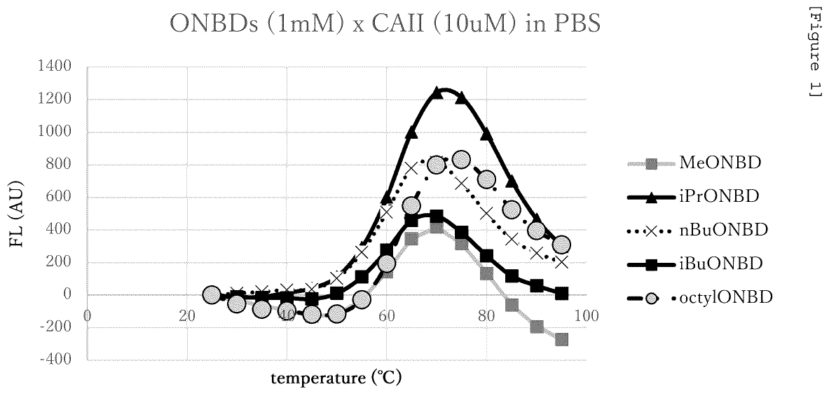

FIG. 1 shows the relationship between temperature and fluorescence intensity. As shown in FIG. 1, when any of the ONBD compounds was added, an increase in the fluorescence intensity was observed from around 60° C. at which heat denaturation of bovine carbonic anhydrase occurred. In particular, the increase in the fluorescence intensity was significant when iPrONBD was added. These results suggest that it is possible to quantify the degree of heat denaturation of proteins (visualization of the denatured state of proteins) by using ONBD compounds.

### [Example 3] Quantification of aggregated state of aggregating proteins

### 3-1. Amyloid β1-40

Amyloid β1-40 (Peptide Institute, Inc., Trifluoroacetate Form, 4307-v) was diluted with a buffer (100 mM NaCl, 20 mM Tris, pH: 7.4) to a final concentration of 100 μM, and a solution was prepared from a 10 mM DMSO solution of 100 mM ONBD compound and Thioflavin T so that the final concentration of the fluorescent compound was 1 mM. 50 μL of the solution was added to each well of a 96-well half well black plate (Greiner, 675086), and the fluorescence (using filters for Ex: Em=485+20/530±20 nm) was measured in a plate reader (Teccan, Infinite 200 Pro F PLEX) under constant temperature conditions at 37° C. with plate shaking at 1 second per minute. The ONBD compounds used were the following three types: MeONBD, iPrONBD, and octylONBD, described above.

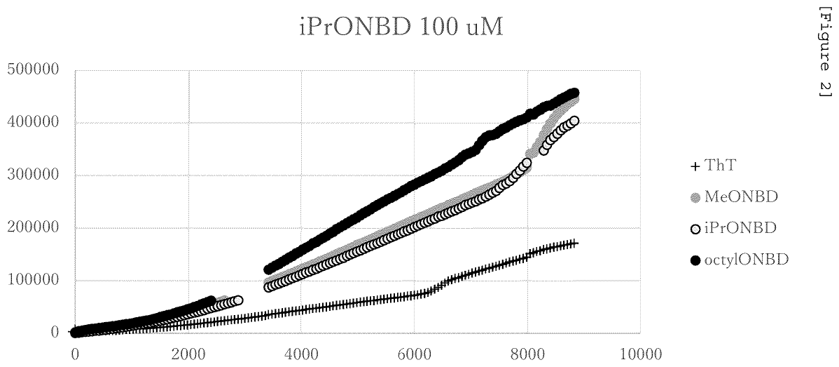

FIG. 2 shows the relationship between time from the start of measurement and fluorescence intensity. As shown in FIG. 2, when any of the ONBD compounds was added, the fluorescence intensity was increased as time passed, and the increase was much larger than that of Thioflavin T, which is a known amyloid S detection dye. These results suggest that it is possible to quantify protein aggregation (visualization of the aggregated state of proteins) by using ONBD compounds.

### 3-2. α-Synuclein

α-Synuclein fibril (Cosmo Bio Co., Ltd., SYN03) was diluted with 10 mM MES buffer (pH: 7.4) to prepare a 500 nM solution. The solution was dispensed in 50 μL portions in a 96-well half well black plate (Greiner, 675086). A stock solution (DMSO solution) of 100 mM ONBD compound and Thioflavin T was once diluted to 1 mM with the above buffer and added to each well to a final concentration of 10 M, followed by pipetting. The fluorescence (using filters for Ex: Em=485120/530±20 nm) was measured in a plate reader (Teccan, Infinite 200 Pro F PLEX) under constant temperature conditions at 37° C. with plate shaking at 1 second per minute. The ONBD compounds used were three types: iPrONBD and octylONBD described above, as well as PEGONBD shown below.

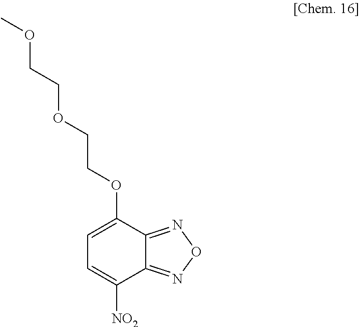 [SMILES: COCCOCCOc1ccc([N+](=O)[O-])c2nonc12]

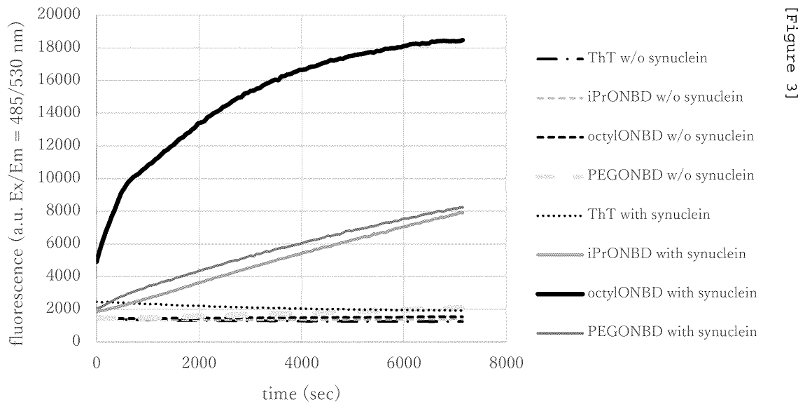

FIG. 3 shows the relationship between time from the start of measurement and fluorescence intensity in the presence or absence of α-synuclein fibril. As shown in FIG. 3, when any of the ONBD compounds was added, the fluorescence intensity was increased as time passed in the presence of α-synuclein fibril. In particular, the increase in the fluorescence intensity was significant when octylONBD was added. These results suggest that it is possible to quantify protein aggregation (visualization of the aggregated state of proteins) by using ONBD compounds.

### [Example 4] Detection of heat-denatured proteins by two-dimensional electrophoresis

A disruption solution of HEK293FT cells solubilized with 1× RIPA buffer (Nacalai, 08714-04) and bovine carbonic anhydrase (hereinafter referred to as “CAII”) (Aldrich, C3934) were diluted with the same buffer to prepare a protein mixed solution in which the protein concentration of the cell disruption solution was 3.0 mg/mL and the CAII concentration was 1 μM or 5 μM. A 1 mM DMSO solution of brinzolamide (TCI, B4258) was added thereto to 1 equivalent of CAII, and the mixture was allowed to stand at room temperature for 30 minutes. For the comparison sample, the same amount of DMSO was added, and the same operation was carried out. Thereafter, centrifugation was performed at 14,000×g for 10 minutes, and the supernatant was collected. A stock solution (DMSO solution) of 100 mM ONBD compound was added to the supernatant to a final concentration of 1 mM. After gentle stirring with a vortex, 50 μL of the solution was transferred to a PCR tube. The PCR tube was heated in a thermal cycler (Applied Biosystems, Mini Amp Plus) at 65° C. for 5 minutes and then allowed to stand at room temperature. The subsequent protocol to prepare two-dimensional electrophoresis samples was performed in the following two ways.

(Protocol to collect all proteins) A sample was prepared according to the protocol of the ReadyPrep 2-D cleanup kit (Bio-Rad), and the final pellets obtained were dissolved in 15 μL of swelling buffer (containing ampolyte, pH: 3-10) included with the Auto2D Glycine Type Reagent Kit.

(Protocol to collect pellets) The heated sample was centrifuged (at 20,000×g for 20 minutes), the supernatant was discarded, and the remaining pellets were dissolved in 15 μL of swelling buffer (containing ampolyte, pH: 3-10) included with the Auto2D Glycine Type Reagent Kit.

Two-dimensional electrophoresis was performed according to the long-term protocol of Auto 2D Plus (Merck), and fluorescence images of the resulting gel were obtained using a fluorescence imager (Vilver Lourmat, Fusion Solo S). Thereafter, all proteins contained in the gel were stained by silver staining (Nacalai, Sil-Best Stain One, 06865-81) or with Oriole (Bio-Rad, 1610495) according to the manufacturer's protocol, and images were taken in the same manner. The ONBD compound used was iPrONBD described above.

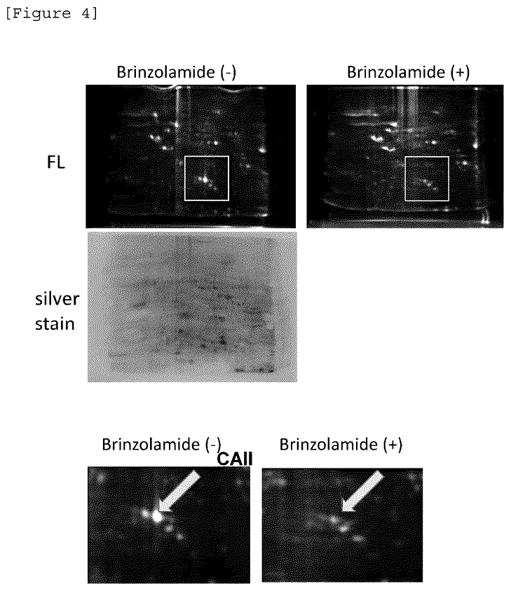

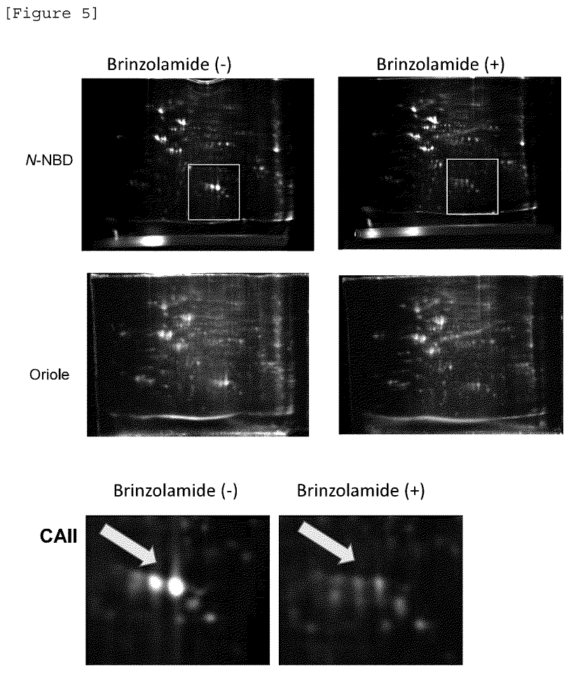

FIG. 4 shows two-dimensional electrophoresis images when samples were prepared according to the protocol to collect all proteins, and FIG. 5 shows two-dimensional electrophoresis images when samples were prepared according to the protocol to collect pellets. As shown in FIGS. 4 and 5, when either of the protocol was used, the fluorescence intensity of CAII was reduced due to the addition of brinzolamide; however, the reduction in the fluorescence intensity was more significant when the protocol to collect pellets was used. The reduction in the fluorescence intensity of CAII due to the addition of brinzolamide is considered to be because CAII acquired resistance to heat denaturation as a result of binding to brinzolamide. These results suggest that even under conditions in which proteins are mixed, it is possible to use ONBD compounds to specify denatured proteins and quantify their degree of denaturation.

### [Example 5] Synthesis of nitrobenzoxadiazole (NBD) derivatives

### 5-1. Cyclo-butyl-ONBD

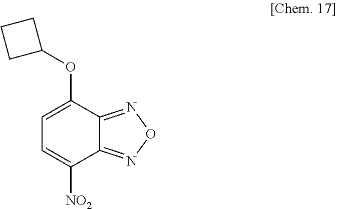 [SMILES: O=[N+]([O-])c1ccc(OC2CCC2)c2nonc12]

F-NBD (TCI, A5593; 20.0 mg, 110 μmol) and cyclobutanol (Angene International Limited, AG002XN2; 1 mL) were added to a ChemiStation (EYELA, Personal Organic Synthesizer) tube (capacity: 15 mL) and reacted at 60° C. for 18 hours. After completion of the reaction, the resultant was dried under reduced pressure and then separated by PTLC (hexane: CHCl₃), thereby obtaining cyclo-butyl-ONBD as a yellow solid (14.0 mg, 54%, 35 μmol). ¹H NMR (600 MHz, CDCl₃) δ_{H} 8.51 (d, J=8.4 Hz, 1H), 6.50 (d, J=8.4 Hz, 1H), 6.50 (quint., J=7.2 Hz, 1H), 2.64-2.59 (m, 2H), 2.46-2.39 (m, 2H), 2.05-1.99 (m, 1H), 1.86-1.78 (m, 1H); ¹³C NMR (150 MHz, CDCl₃) δ_{C} 153.5, 145.4, 144.1, 134.1, 129.7, 105.1, 75.0, 30.2, 13.4; HRMS (ESI, positive): calcd. for [C₁₀H₉N₃O₄+Na]⁺258.0485, found 258.0495.

### 5-2. Cyclo-pentyl-ONBD

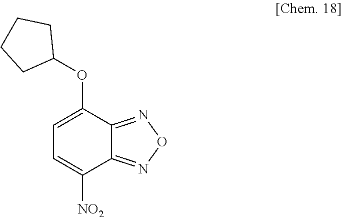 [SMILES: O=[N+]([O-])c1ccc(OC2CCCC2)c2nonc12]

F-NBD (TCI, A5593; 20.0 mg, 110 μmol) and cyclopentanol (Nacalai, 10222-32; 1 mL) were added to a ChemiStation (EYELA, Personal Organic Synthesizer) tube (capacity: 15 mL) and reacted at 60° C. for 24 hours. After completion of the reaction, the resultant was dried under reduced pressure and then separated by PTLC (hexane: CHCl₃), thereby obtaining cyclo-pentyl-ONBD as a yellow solid (19.0 mg, 70%, 76 μmol). ¹H NMR (600 MHz, CDCl₃) δ_{H} 8.53 (d, J=8.4 Hz, 1H), 6.63 (d, J=8.4 Hz, 1H), 5.18 (m, 1H), 2.10-2.07 (m, 4H), 1.91-1.89 (m, 2H), 1.75-1.73 (m, 2H); ¹³C NMR (150 MHz, CDCl₃) δ_{C} 154.5, 145.7, 144.2, 134.2, 129.3, 84.1, 32.9, 24.2; HRMS (ESI, positive): calcd. for [C₁₁H₁₁N₃O₄+Na]⁺272.0642, found 272.0641.

### 5-3. Cyclo-hexyl-ONBD

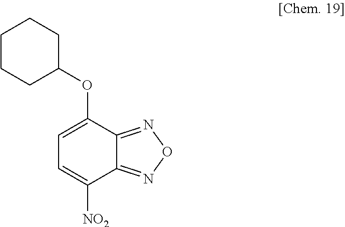 [SMILES: O=[N+]([O-])c1ccc(OC2CCCCC2)c2nonc12]

F-NBD (TCI, A5593; 20.0 mg, 110 μmol) and cyclohexanol (Nacalai, 10111-95; 1 mL) were added to a ChemiStation (EYELA, Personal Organic Synthesizer) tube (capacity: 15 mL) and reacted at 60° C. for 24 hours. After completion of the reaction, the resultant was dried under reduced pressure and then separated by PTLC (hexane: CHCl₃), thereby obtaining cyclo-hexyl-ONBD as a yellow solid (17.0 mg, 60%, 65 μmol). ¹H NMR (600 MHz, CDCl₃) δ_{H} 8.52 (d, 1H, J=8.4 Hz), 6.65 (d, 1H, J=8.4 Hz), 4.78 (m, 1H), 2.13-2.10 (m, 2H), 1.92-1.89 (m, 2H), 1.81-1.75 (m, 2H), 1.66-1.62 (m, 2H), 1.50-1.37 (m, 2H); ¹³C NMR (150 MHz, CDCl₃) δ_{C} 154.3, 145.8, 144.2, 134.3, 129.1, 105.1, 79.8, 31.2, 25.2, 23.5; HRMS (ESI, positive): calcd. for [C₁₂H₁₃N₃O₄+Na]⁺286.0798, found 286.0814.

### 5-4. Cyclo-heptyl-ONBD

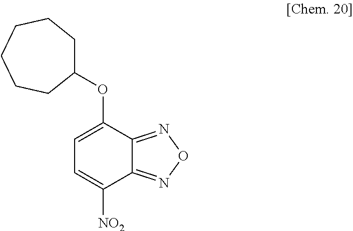 [SMILES: O=[N+]([O-])c1ccc(OC2CCCCCC2)c2nonc12]

F-NBD (TCI, A5593; 20.0 mg, 110 μmol) and cycloheptanol (Angene International Limited, AG0034NE; 1 mL) were added to a ChemiStation (EYELA, Personal Organic Synthesizer) tube (capacity: 15 mL) and reacted at 65° C. for 15 hours. After completion of the reaction, the resultant was dried under reduced pressure and then separated by PTLC (hexane: CH₂Cl₂), thereby obtaining cyclo-heptyl-ONBD as a yellow solid (13.0 mg, 40%, 47 mol). ¹H NMR (600 MHz, CDCl₃) δH 8.53 (d, 1H, J=8.4 Hz), 6.58 (d, 1H, J=8.4 Hz), 4.91 (m, 1H), 2.18 (m, 2H), 2.01 (m, 2H), 1.81 (m, 2H), 1.66 (m, 4H), 1.54 (m, 2H); ¹³C NMR (150 MHz, CDCl₃) δ_{C} 154.3, 145.8, 144.2, 134.3, 129.1, 105.1, 82.5, 33.4, 28.3, 22.8; HRMS (ESI, positive): calcd. for [C₁₋₃H₁₅N₃O₄+Na]⁺300.0955, found 300.0978.

### 5-5. Cyclo-octyl-ONBD

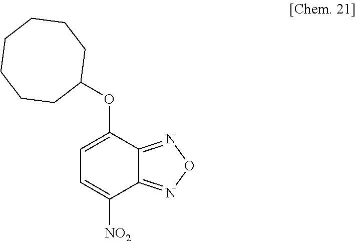 [SMILES: O=[N+]([O-])c1ccc(OC2CCCCCCC2)c2nonc12]

F-NBD (TCI, A5593; 20.0 mg, 110 μmol) and cyclooctanol (TCI, C0623; 1 mL) were added to a microwave (Biotage, Initiator Plus Microwave Synthesizer) vial (capacity: 0.5-2 mL) and reacted at 150° C. for 1 hour. After completion of the reaction, the resultant was dried under reduced pressure and then separated by PTLC (hexane: CH₂Cl₂), thereby obtaining cyclo-octyl-ONBD as a yellow solid (10.0 mg, 31%, 34 μmol). ¹H NMR (600 MHz, CDCl₃) δ_{H} 8.52 (d, J=7.8 Hz, 1H), 6.57 (d, J=7.8 Hz, 1H), 4.94-4.90 (m, 1H), 2.12-2.04 (m, 4H), 1.90-1.83 (m, 2H), 1.72-1.49 (m, 8H); ¹³C NMR (150 MHz, CDCl₃) δ_{C}154.2, 145.8, 144.2, 134.4, 129.0, 105.1, 82.8, 31.5, 26.9, 25.7, 23.1; HRMS (ESI, positive): calcd. for [C₁₄H₁₇N₃O₄+Na]⁺314. 1111, found 314.1112.

### 5-6. 2-Adamantyl-ONBD

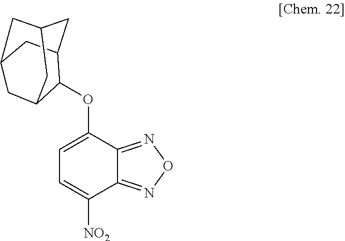 [SMILES: O=[N+]([O-])c1ccc(OC2C3CC4CC(C3)CC2C4)c2nonc12]

F-NBD (TCI, A5593; 20.0 mg, 110 μmol), 2-adamantanol (TCI, A0720; 1 mL), and DMF (Wako, 045-02911; N,N-dimethylformamide, 3 mL) were added to a microwave (Biotage, Initiator Plus Microwave Synthesizer) vial (capacity: 0.5-2 mL) and reacted at 150° C. for 1 hour. After completion of the reaction, the resultant was dried under reduced pressure and then separated by PTLC (hexane: CHCl₃), thereby obtaining 2-adamantyl-ONBD as a yellow solid (10.0 mg, 31%, 34 μmol). ¹H NMR (600 MHz, CDCl₃) δ_{H} 8.51 (d, J=8.4 Hz, 1H), 6.64 (d, J=8.4 Hz, 1H), 4.94 (s, 1H), 2.33 (s, 2H), 2.22 (d, J=12 Hz, 2H), 2.02-1.94 (m, 4H), 1.86-1.81 (m, 4H), 1.66 (d, J=12 Hz, 1H); ¹³C NMR (150 MHz, CDCl₃) δ_{C} 154.2, 145.9, 144.3, 134.3, 129.1, 105.4, 84.2, 37.1, 36.3, 31.6, 31.5, 27.0, 26.9

### 5-7._n-Hexyl-ONBD

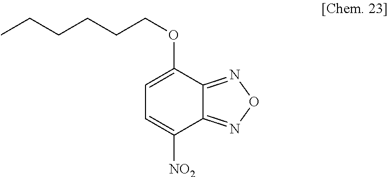 [SMILES: CCCCCCOc1ccc([N+](=O)[O-])c2nonc12]

F-NBD (TCI, A5593; 20.0 mg, 110 μmol) and 1-hexanol (Nacalai, 18013-45; 1 mL) were added to a microwave (Biotage, Initiator Plus Microwave Synthesizer) vial (capacity: 0.5-2 mL) and reacted at 150° C. for 1 hour. After completion of the reaction, the resultant was dried under reduced pressure and then separated by PTLC (hexane: CHCl₃), thereby obtaining n-hexyl-ONBD as a yellow solid (20.0 mg, 69%, 75 μmol). ¹H NMR (600 MHz, CDCl₃) δ_{H} 8.53 (d, J=8.7 Hz, 1H), 6.65 (d, J=8.7 Hz, 1H), 4.37 (t, J=6.6 Hz, 2H), 2.00-1.97 (m, 2H), 1.56-1.50 (m, 2H), 1.40-1.32 (m, 4H), 0.91 (t, J=7.2 Hz, 3H); ¹³C NMR (150 MHz, CDCl₃) δ_{C} 155.2, 145.4, 144.1, 134.2, 129.7, 104.3, 71.5, 31.4, 28.6, 25.6, 22.6, 14.0; HRMS (ESI, positive): calcd. for [C₁₂H₁₅N₃O₄+Na]⁺288.0955, found 288.0974.

### 5-8. n-Octyl-ONBD

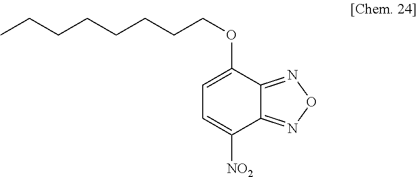 [SMILES: CCCCCCCCOc1ccc([N+](=O)[O-])c2nonc12]

F-NBD (TCI, A5593; 20.0 mg, 110 μmol) and 1-octanol (TCI, 00036; 1 mL) were added to a microwave (Biotage, Initiator Plus Microwave Synthesizer) vial (capacity: 0.5-2 mL) and reacted at 160° C. for 1 hour. After completion of the reaction, the resultant was dried under reduced pressure and then separated by PTLC (hexane: CHCl₃), thereby obtaining n-octyl-ONBD as a yellow solid (20.0 mg, 62%, 68 μmol). ¹H NMR (600 MHz, CDCl₃) δ_{H} 8.52 (d, J=8.4 Hz, 1H), 6.65 (d, J=8.4 Hz, 1H), 4.37 (t, J=7.2 Hz, 2H), 2.00-1.92 (m, 2H), 1.57-1.50 (m, 2H), 1.40-1.25 (m, 8H), 0.87 (t, J=6.6 Hz, 3H); ¹³C NMR (150 MHz, CDCl₃) δ_{C} 155.2, 145.2, 144.1, 134.1, 129.7, 104.3, 71.5, 32.0, 29.7, 29.6, 29.5, 29.4, 29.3, 25.9, 22.8, 14.2; HRMS (ESI, positive): calcd. for [C₁₄H₁₉N₃O₄+Na]⁺316.1262, found 316.1268.

### 5-9. n-Decyl-ONBD

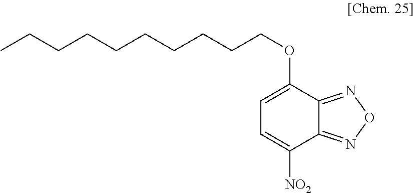 [SMILES: CCCCCCCCCCOc1ccc([N+](=O)[O-])c2nonc12]

F-NBD (TCI, A5593; 20.0 mg, 110 μmol) and 1-decanol (Nacalai, 10618-62; 1 mL) were added to a ChemiStation (EYELA, Personal Organic Synthesizer) tube (capacity: 15 mL) and reacted at 60° C. for 16 hours. After completion of the reaction, the resultant was dried under reduced pressure and then separated by PTLC (hexane: CHCl₃), thereby obtaining n-decyl-ONBD as a yellow solid (26.3 mg, 75%, 81.8 μmol). ¹H NMR (600 MHz, CDCl₃) δ_{H} 8.52 (d, J=8.1 Hz, 1H), 6.65 (d, J=8.1 Hz, 1H), 4.73 (t, J=6.6 Hz, 2H), 2.02-1.93 (m, 2H), 1.56-1.50 (m, 2H), 1.40-1.26 (m, 12H), 0.86 (t, J=7.2 Hz, 3H); ¹³C NMR (150 MHz, CDCl₃) δ_{C} 155.2, 145.4, 144.1, 134.1, 129.7, 104.3, 71.5, 32.0, 29.7, 29.6, 29.5, 29.4, 29.3, 28.6, 25.9, 22.8, 14.2; HRMS (ESI, positive): calcd. for [C₁₆H₂₃N₃O₄+Na]⁺344.1589, found 344.1581.

### 5-10. n-Dodecyl-ONBD

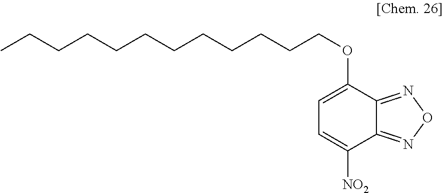 [SMILES: CCCCCCCCCCCCOc1ccc([N+](=O)[O-])c2nonc12]

F-NBD (TCI, A5593; 20.0 mg, 110 μmol) and 1-dodecanol (Nacalai, 20118-15; 1 mL) were added to a microwave (Biotage, Initiator Plus Microwave Synthesizer) vial (capacity: 0.5-2 mL) and reacted at 170° C. for 1 hour. After completion of the reaction, the resultant was dried under reduced pressure and then separated by PTLC (hexane: CHCl₃), thereby obtaining n-dodecyl-ONBD as a yellow solid (20.0 mg, 52%, 57 μmol). ¹H NMR (600 MHz, CDCl₃) δ_{H} 8.53 (d, J=8.1 Hz, 1H), 6.65 (d, J=8.1 Hz, 1H), 4.37 (t, J=7.2 Hz, 2H), 2.03-1.95 (m, 2H), 1.55-1.50 (m, 2H), 1.40-1.36 (m, 2H), 1.33-1.25 (m, 14H), 0.87 (t, J=7.2 Hz, 3H); ¹³C NMR (150 MHz, CDCl₃) δ_{C} 155.2, 145.4, 144.1, 134.1, 129.7, 104.3, 71.5, 32.0, 29.7, 29.7, 29.6, 29.5, 29.4, 29.3, 28,6, 25.9, 22.8, 14.2; HRMS (ESI, positive) calcd. for [C₁₈H₂₇N₃O₄+Na]⁺372.1894, found 372.1896.

### 5-11. i-Butyl-ONBD

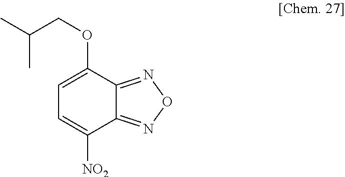 [SMILES: CC(C)COc1ccc([N+](=O)[O-])c2nonc12]

F-NBD (TCI, A5593; 25.0 mg, 140 μmol), 2-methylpropan-1-ol (TCI, 310 μL, 3.40 mmol), DIEPA (Nacalai, 14014-42; N-ethyl-N-isopropylpropan-2-amine, 138 μL, 140 μmol), and CH₂Cl₂ (Nacalai, 22430-74; 3.8 mL) were added to a ChemiStation (EYELA, Personal Organic Synthesizer) tube (capacity: 5 mL) and reacted at room temperature for 24 hours. After completion of the reaction, the resultant was dried under reduced pressure and then separated by column chromatography (hexane: AcOEt), thereby obtaining i-butyl-ONBD as a yellow solid (22.3 mg, 69%, 94.0 μmol). ¹H NMR (600 MHz, CDCl₃) δ_{H} 8.53 (d, J=8.4 Hz, 1H), 6.65 (d, J=8.4 Hz, 1H), 4.13 (d, J=6.6 Hz, 2H), 2.33-2.29 (m, 1H) 1.15-1.09 (m, 6H); ¹³C NMR (150 MHz, CDCl₃) δ_{C} 155.3, 145.4, 144.1, 134.2, 129.7, 104.4, 28.1, 19.1; HRMS (ESI, positive): calcd. for [C₁₀H₁₁N₃O₄+Na]⁺260.0642, found 260.0653.

### 5-12. 5-Nonyl-ONBD

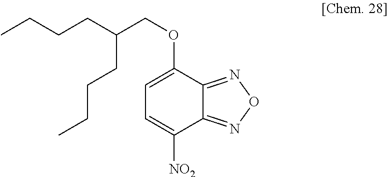 [SMILES: CCCCC(CCCC)COc1ccc([N+](=O)[O-])c2nonc12]

F-NBD (TCI, A5593; 20.0 mg, 110 μmol) and cyclooctanol (TCI, C0623; 1 mL) were added to a microwave (Biotage, Initiator Plus Microwave Synthesizer) vial (capacity: 0.5-2 mL) and reacted at 150° C. for 1 hour. After completion of the reaction, the resultant was dried under reduced pressure and then separated by PTLC (hexane: CHCl₃), thereby obtaining 5-nonyl-ONBD as a yellow solid (10.0 mg, 31%, 34 μmol). ¹H NMR (600 MHz, CDCl₃) δ_{H} 8.53 (d, J=8.7 Hz, 1H), 6.62 (d, J=8.7 Hz, 1 H), 4.79-4.72 (m, 1H), 1.90-1.77 (m, 4H), 1.47-1.31 (m, 8H), 0.89 (t, J=7.2 Hz, 6H); ¹³C NMR (150 MHz, CDCl₃) δ_{C}155.1, 145.7, 144.3, 134.3, 129.1, 104.9, 82.8, 33.4, 27.5, 22.7, 14.0; HRMS (ESI, positive): calcd. for [C₁₅H₂₁N₃O₄+Na]⁺330. 1424, found 330. 1440.

### 5-13. Br-NBD (N25_044)

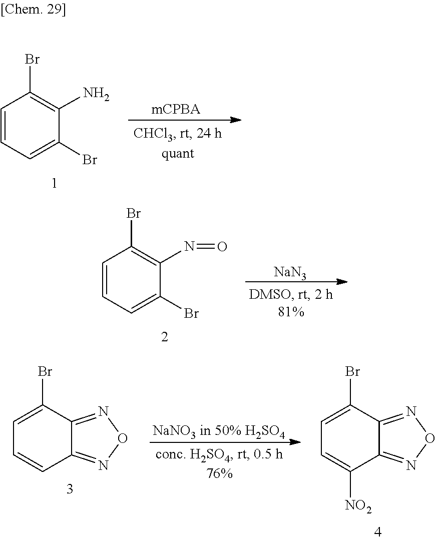

### 1,3-Dibromo-2-nitrosobenzene (2)

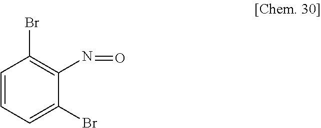

2,6-Dibromoaniline (BLD Pharmatech Ltd., BD34272; 500 mg, 1.99 mmol), 75% mCPBA (Oakwood Products, Inc., 080350; 3-Choloroperbenzonic acid 75%, 458 mg, 1.99 mmol), and CHCl₃ (Nacalai, 08401-94; 3.8 mL) were added to a round-bottom flask (capacity: 50 mL) and reacted at room temperature for 24 hours. After completion of the reaction, the resultant was diluted with CHCl₃ and washed with a saturated NaS₂O₄ aqueous solution, saturated NaHCO₃, and saturated brine, and the organic layer was dried over Na₂SO₄ and filtered. The filtrate was dried under reduced pressure, after which a brown solid of 1,3-dibromo-2-nitrosobenzene (2) was used in the second-step reaction without purification.

### 4-Bromobenzo[c][1,2,5]oxadiazole (3)

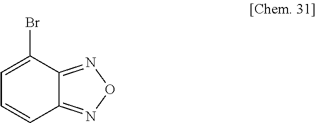

2 (500 mg, 1.89 mmol), NaN₃ (Nacalai, 31208-82; 135 mg, 2.08 mmol), and DMSO (Wako, 048-32811; 10 mL) were added to a round-bottom flask (capacity: 50 mL) and reacted at room temperature for 2 hours. After completion of the reaction, the solution was heated to 120° C. and poured into ice water, and the precipitated solid was filtered and dried, after which 4-bromobenzo[c][1,2,5]oxadiazole (3) was obtained as a brown solid (300 mg, 81%, 1.52 mmol). ¹H NMR (600 MHz, CDCl₃) δ_{H} 7.81 (d, J=7.8 Hz, 1H), 7.63 (d, J=7.8 Hz, 1H), 7.29 (m, 1H).

### Br-NBD (4)

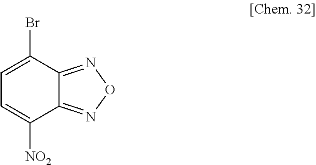 [SMILES: O=[N+]([O-])c1ccc(Br)c2nonc12]

In a round-bottom flask (capacity: 50 mL), 3 (161 mg, 809 μmol) was dissolved in H₂SO₄ (Nacalai, 32519-95; 1.3 mL), and NaNO₃ (Nacalai, 31916-15; 89.4 mg, 1.05 mmol) was added dropwise to 50% H₂SO₄ (1 mL) and reacted at room temperature for 30 minutes. After completion of the reaction, the solution was heated to 85° C. and poured into ice water, and the precipitated solid was filtered, dried, and then separated by column chromatography (hexane: AcOEt), thereby obtaining Br-NBD as a brown solid (150 mg, 76%, 615 μmol). ¹H NMR (600 MHz, CDCl₃) δ_{H} 8.37 (d, J=7.8 Hz, 1H), 7.86 (d, J=7.8 Hz, 1H); ¹³C NMR (150 MHz, CDCl₃) δ_{C} 150.6, 142.4, 136.3, 132.3, 130.6, 119.2.

### 5-14. I-NBD

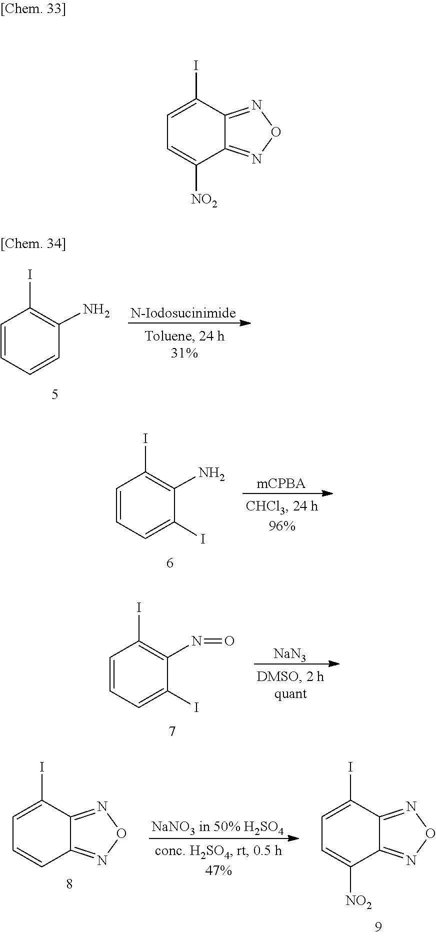

### 2,6-Diiodoaniline (6)

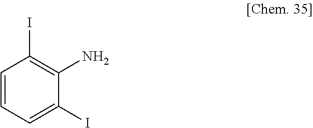

2-Iodoaniline (TCI, 10046; 50 mg, 230 μmol), N-iodosuccinimide (Nacalai, 19331-74; 51 mg, 230 μmol), acetic acid (Nacalai, 00211-95; 13 μL, 230 μmol), and toluene (Wako, 204-01861; 3.8 mL) were added to a round-bottom flask (capacity: 50 mL) and reacted at room temperature for 24 hours. After completion of the reaction, the resultant was dried under reduced pressure and then separated by column chromatography (hexane: AcOEt), thereby obtaining 2,6-diiodoaniline (6) as a brown solid (31 mg, 39%, 90 μmol). ¹H NMR (600 MHz, CDCl₃) δ_{H} 7.61 (d, J=7.8 Hz, 2H), 6.14 (t, J=7.8 Hz, 1H), 4.60 (s, 2H); ¹³C NMR (150 MHz, CDCl₃) δ_{C} 146.2, 139.5, 121.3, 81.6.

### 1,3-Diiodo-2-nitrosobenzene (7)

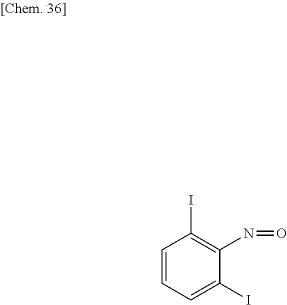

Crude 6 (31 mg, 90 μmol), 75% mCPBA (Oakwood Products, Inc., 080350; 3-Choloroperbenzonic acid 75%, 47 mg, 270 μmol), and CHCl₃ (Nacalai, 08401-94; 6 mL) were added to a round-bottom flask (capacity: 50 mL) and reacted at room temperature for 24 hours. After completion of the reaction, the resultant was diluted with CHCl₃ and washed with a saturated NaS₂O₄ aqueous solution, saturated NaHCO₃, and brine, and the organic layer was dried over Na₂SO₄ and filtered. The filtrate was dried under reduced pressure, after which 1,3-diiodo-2-nitrosobenzene (7) was obtained as a brown solid (31 mg, 96%, 86 μmol). ¹H NMR (600 MHz, CDCl₃) δ_{H} 7.61 (d, J=8.1 Hz, 2H), 6.15 (d, J=8.1 Hz, 1H).

### 4-Iodobenzo[c][1,2,5]oxadiazole (8)

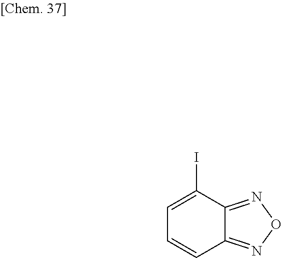

7 (31 mg, 86 μmol), NaN₃ (Nacalai, 31208-82; 6.2 mg, 95 μmol), and DMSO (Wako, 048-32811; 3 mL) were added to a round-bottom flask (capacity: 30 mL) and reacted at room temperature for 2 hours. After completion of the reaction, the solution was heated to 120° C. and poured into ice water, and the precipitated solid was filtered and dried, after which a brown solid of 4-iodobenzo[c][1,2,5]oxadiazole (8) was used in the fourth-step reaction without purification.

### I-NBD (9)

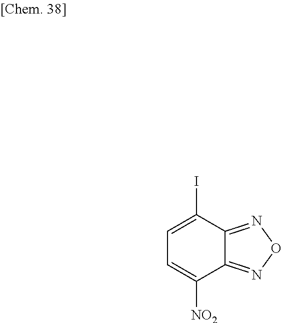 [SMILES: O=[N+]([O-])c1ccc(I)c2nonc12]

In a round-bottom flask (capacity: 30 mL), 8 (31 mg, 130 mmol) was dissolved in H₂SO₄ (Nacalai, 32519-95; 1.3 mL), and a mixed liquid of 50% H₂SO₄ (1 mL) and NaNO₃ (Nacalai, 31916-15; 14 mg, 160 mmol) was added dropwise and reacted at room temperature for 30 minutes. After completion of the reaction, the solution was heated to 85° C. and poured into ice water, and the precipitated solid was filtered, dried, and then separated by column chromatography (hexane: AcOEt), thereby obtaining I-NBD as a brown solid (10 mg, 27%, 34 μmol). ¹H NMR (600 MHz, CDCl₃) δ_{H} 8.20 (d, J=7.5 Hz, 1H), 8.14 (d, J=7.5 Hz, 1H); ¹³C NMR (150 MHz, CDCl₃) δ_{C} 152.7, 141.0, 139.3, 130.2, 90.7.

### 5-15. Diethylenegylycol-ONBD (DEG-ONBD)

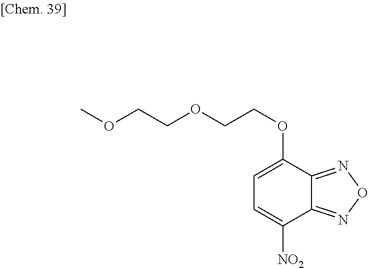 [SMILES: COCCOCCOc1ccc([N+](=O)[O-])c2nonc12]

F-NBD (TCI, A5593; 48.0 mg, 260 μmol), 2-(2-methoxyethoxy)ethanol (TCI, M0537; 15 μL, 130 μmol), DIEPA (Nacalai, 14014-42; N-ethyl-N-isopropylpropan-2-amine, 89 μL, 510 μmol), and CH₂Cl₂ (Nacalai, 22430-74; 1.5 mL) were added to a ChemiStation (EYELA, Personal Organic Synthesizer) tube (capacity: 15 mL) and reacted at 0° C. for 1 hour. After completion of the reaction, the resultant was dried under reduced pressure and then separated by column chromatography (hexane: AcOEt), thereby obtaining diethylenegylycol-ONBD as a brown amorphous solid (27.6 mg, 37%, 97.4 μmol). ¹H NMR (600 MHz, CDCl₃) δ_{H} 8.52 (d, J=9.0 Hz, 1H), 6.76 (d, J=9.0 Hz, 1H), 4.58 (t, J=4.6 Hz, 2H), 4.02 (t, J=4.6 Hz, 2H), 3.74 (t, J=4.6 Hz, 2H), 3.56 (t, J=4.6 Hz, 2H), 3.36 (s, 3H); HRMS (ESI, positive): calcd. for [C₁₁H₁₃N₃O₆+Na]⁺306.0697, found 306.0726.

### [Example 6] Synthesis of dimethylaminosulfonyl benzoxadiazole (DBD) derivative

### 6-1. Me-ODBD

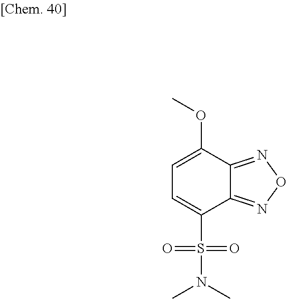 [SMILES: COc1ccc(S(=O)(=O)N(C)C)c2nonc12]

F-DBD (TCI, A5595; 15.0 mg, 61 μmol), methanol (Nacalai, 21914-74; 62 μL, 1.5 mmol), sodium hydride (Nacalai, 31525-22; 1.5 mg, 61 μmol), and DMF (Wako, 045-02911; N,N-dimethylformamide, 3 mL) were added to a ChemiStation (EYELA, Personal Organic Synthesizer) tube (capacity: 15 mL) and reacted at room temperature for 24 hours. After completion of the reaction, the resultant was dried under reduced pressure and then separated by column chromatography (hexane: AcOEt), thereby obtaining Me-ODBD as a yellow solid (15 mg, 95%, 58 μmol). ¹H NMR (600 MHz, CDCl₃) δ_{H} 7-99 (d, J=7-8 Hz, 1H, 6.61 d=7.8 Hz, 1H), 4.14 (s, 3H), 2.91 (s, 6H); 13C NMR (150 MHz, CDCl₃) δ_{C} 152.7, 146.6, 145.0, 137.4, 118.1, 104.4, 57.2, 37.8; HRMS (ESI, positive): calcd. for [C₉H₁₁N₃O₄S+Na]⁺280. 0362, found 280. 0377.

### 6-2. i-Pr-ODBD

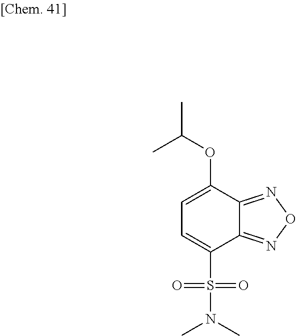 [SMILES: CC(C)Oc1ccc(S(=O)(=O)N(C)C)c2nonc12]

F-DBD (TCI, A5595; 15 mg, 61 μmol), 2-propanol (Wako, 165-09161; 120 μL, 1.5 mmol), sodium hydride (Nacalai, 31525-22; 1.5 mg, 61 μmol), and DMF (Wako, 045-02911; N,N-dimethylformamide, 3 mL) were added to a ChemiStation (EYELA, Personal Organic Synthesizer) tube (capacity: 15 mL) and reacted at room temperature for 24 hours. After completion of the reaction, the resultant was dried under reduced pressure and then separated by column chromatography (hexane: AcOEt), thereby obtaining i-Pr-ODBD as a yellow solid (8.5 mg, 49%, 30.0 μmol). 1H NMR (600 MHz, CDCl₃) δ_{H} 7.96 (d, J=8.1 Hz, 1H), 6.58 (d, J=8.1 Hz, 1H), 4.97-4.91 (m, 1H), 2.97-2.87 (m, 6H) 1.52 (d, J=6.0 Hz, 6H); ¹³C NMR (150 MHz, CDCl₃) δ_{C}151.3, 146.8, 145.5, 137.7, 117.1, 105.5, 73.6, 37.8, 21.7; HRMS (ESI, positive): calcd. for [C₁₁H₁₅N₃O₄S+Na]⁺308.0675, found 308.0692.

### [Example 7] NBD derivatives and probe characteristics for the detection of heat denaturation of carbonic anhydrase

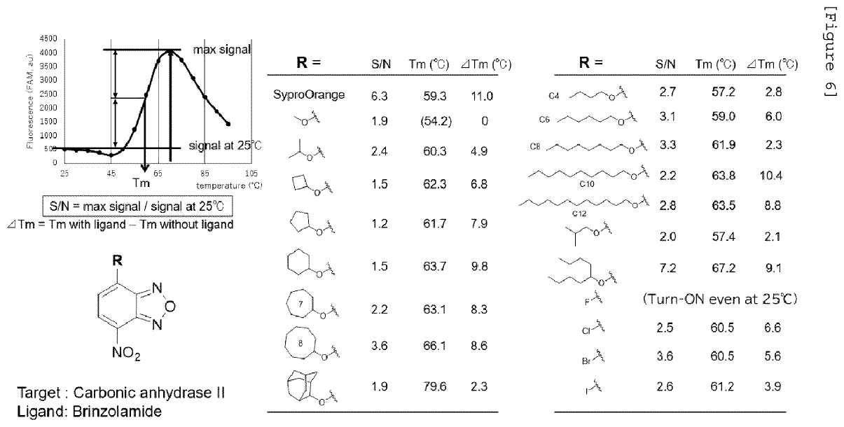

An NBD derivative or Sypro Orange was added to carbonic anhydrase II and heated in the presence or absence of brinzolamide, which is an inhibitor for carbonic anhydrase II. Example 5 shows the synthesis method of novel compounds among the NBD derivatives used in the experiment. Changes in the fluorescence intensity with heating were measured, and S/N, Tm, and ΔTm of each NBD derivative and Sypro Orange were determined (FIG. 6). Tm was defined as the temperature showing the average of the fluorescence intensity at 25° C. and the maximum fluorescence intensity. S/N and ΔTm were calculated from the following formulas.

S/N=maximum fluorescence intensity/fluorescence intensity at 25° C.

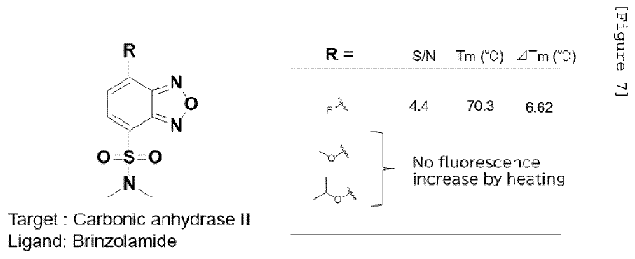

ΔTm=Tm in the presence of brinzolamide−Tm in the absence of brinzolamide

[Example 8] DBD derivatives and probe characteristics for the detection of heat denaturation of carbonic anhydrase A DBD derivative was added to carbonic anhydrase II and heated in the presence or absence of brinzolamide, which is an inhibitor for carbonic anhydrase II. Example 6 shows the synthesis method of novel compounds among the DBD derivatives used in the experiment. Changes in the fluorescence intensity with heating were measured, and S/N, Tm, and ΔTm of each DBD derivative were determined in the same manner as in Example 7 (FIG. 7).

### [Example 9] Fluorescent imaging of intracellular denatured proteins

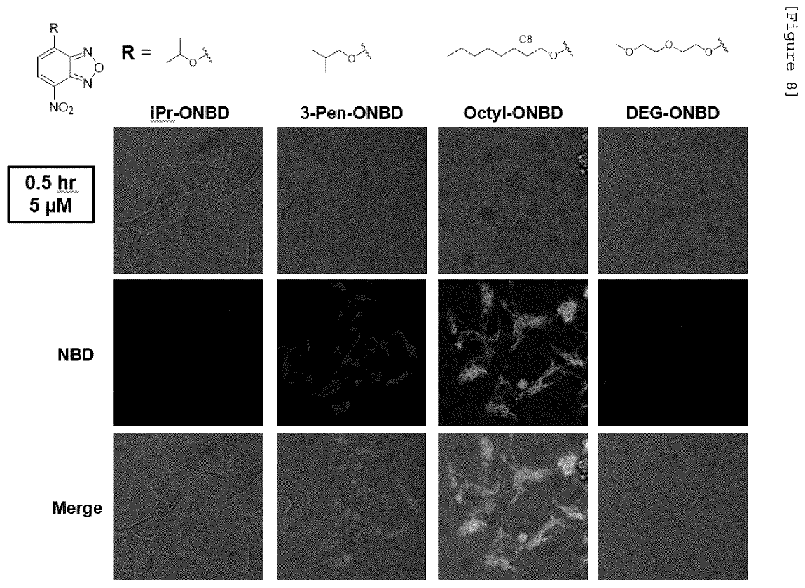

HEK293T cells were seeded (8×10⁴ cells/mL, 2 mL) on a glass base dish (IWAKI 3910-035) coated with a poly-L-lysine solution (Sigma-Aldrich P4707) and cultured overnight, and then 1 μL of DMSO solution of 10 mM ONBD compound was added (final concentration: 5 μM). After treatment in a 37° C. CO₂ incubator for 30 minutes, the cells were observed under a confocal laser fluorescence microscope (ZEISS LSM900, Ex 488 nm/Em 400-550 nm),and images were obtained. FIG. 8 shows images when using iPr-ONBD, 3-Pen-ONBD, Octyl-ONBD, or DEG-ONBD as the ONBD compound.

### [Example 10] Fluorescent imaging under proteasome inhibitor treatment conditions (simultaneous treatment with ONBD compound)

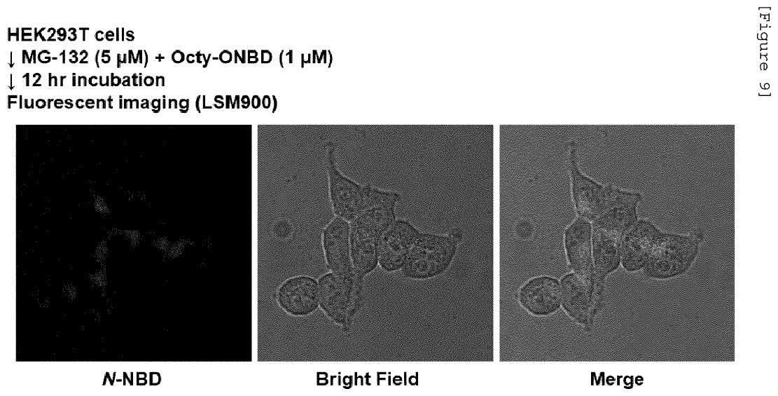

HEK293T cells were seeded (1×10⁵ cells/mL, 2 mL) on a glass base dish (IWAKI 3910-035) coated with a poly-L-lysine solution (Sigma-Aldrich P4707) and cultured overnight, and then 2 μL (final concentration: 5 μM) of DMSO solution of 5 mM MG-132 and 1 μL of DMSO solution of 2 mM OctylNBD compound were added (final concentration: 1 M). After treatment in a 37° C. CO₂ incubator for 12 hours, the cells were observed under a confocal laser fluorescence microscope (ZEISS LSM900, Ex 488 nm/Em 400-550 nm),and images were obtained (FIG. 9). Due to the addition of the proteasome inhibitor, intracellular ubiquitinated proteins, which should be naturally degraded, accumulate without being degraded. Ubiquitinated denatured proteins are considered to accumulate and stain intracellularly.

### [Example 11] Fluorescent imaging under proteasome inhibitor treatment conditions (post-treatment with ONBD compound)

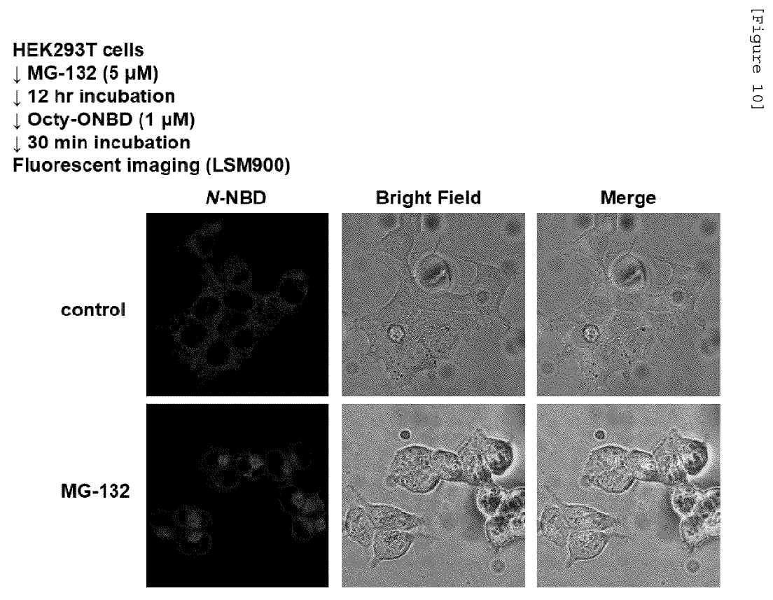

HEK293T cells were seeded (1×10⁵ cells/mL, 2 mL) on a glass base dish (IWAKI 3910-035) coated with a poly-L-lysine solution (Sigma-Aldrich P4707) and cultured overnight, and then 2 μL (final concentration: 5 μM) of DMSO solution of 5 mM MG-132 (proteasome inhibitor) was added and treated in a CO₂ incubator for 12 hours. Then, 1 μL of DMSO solution of 2 mM OctylNBD compound was added, and after incubation for 30 minutes (final concentration: 1 μM), the cells were observed under a confocal laser fluorescence microscope (ZEISS LSM900, Ex 488 nm/Em 400-550 nm),and images were obtained (FIG. 10).

### [Example 12] Quantification of aggregated state of amyloid β1-40

Amyloid β1-40 (Peptide Institute, Inc., Trifluoroacetate Form, 4307-v) was diluted with a buffer (150 mM NaCl, 20 mM Tris, pH: 7.4) to a final concentration of 100 M. A solution was prepared from a DMSO solution of 10 mM probe compound or Thioflavin T so that the final concentration of the fluorescence compound was 100 M. 50 μL of the solution was added to each well of a 96-well half well black plate (Greiner, 675086), and the fluorescence (using filters for Ex: Em=485±20/530±20 nm) was measured in a plate reader (Teccan, Infinite 200 Pro F PLEX) under constant temperature conditions at 37° C. with plate shaking at 1 second per minute.

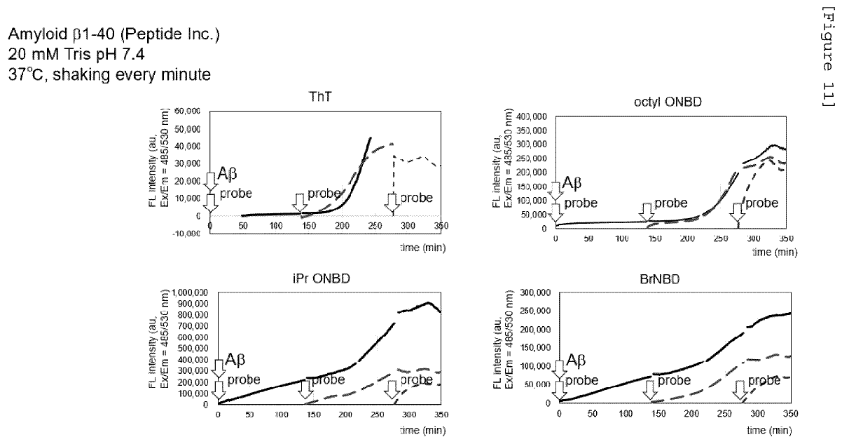

In addition, a probe compound was added to aggregated amyloid β1-40. In this case, the above operation was carried out without adding a probe compound to a diluted solution of amyloid β1-40, after a certain period of incubation time, a DMSO solution of 10 mM probe compound or Thioflavin T was added so that the final concentration of the fluorescence compound was 100 μM, and the fluorescence measurement in the plate reader was continued. FIG. 11 shows changes in the fluorescence intensity over time when using Thioflavin T and when using octyl ONBD, iPr ONBD, or BrNBD as the probe compound.

### [Example 13] Preparation of mass spectrometry samples for detection of labeled sites

Bovine carbonic anhydrase (Aldrich, C3934) was diluted with 10 mM PBS to a final concentration of 10 μM. A 1 mM DMSO solution of brinzolamide (TCI, B4258) was added to 1 equivalent of protein, and the mixture was allowed to stand at room temperature for 30 minutes. For the comparison target, the same amount of DMSO was added, and the same operation was carried out. Subsequently, a stock solution (DMSO solution) of 100 mM ONBD compound was added to a final concentration of 1 mM, and after gentle stirring with a vortex, 50 μL of the solution was transferred to a PCR tube. The PCR tube was heated at each temperature for 5 minutes using a thermal cycler (Applied Biosystems, Mini Amp Plus) as a precision thermocycler. 5× SDS-PAGE sample buffer was added to the reaction solution (final conc. 50 mM Tris-HCl, pH: 6.8, 125 mM 2-mercaptoethanol, 2% sodium dodecyl sulfate (SDS), 0.025% bromophenol blue, 10% glycerol) and heated at 95° C. for 5 minutes. Thereafter, SDS-PAGE was performed using 4-20% acrylamide gel (Bio-Rad). After the fluorescence of the resulting gel was detected with a fluorescence imager (Vilver Lourmat, Fusion Solo S), proteins contained in the gel were CBB-stained. The band corresponding to the protein to be analyzed was separated, and the cut-out band was cut (about 1-mm pieces). The gel pieces were transferred to microtubes, 1 mL of water was added and incubated at 37° C. for 10 minutes, and then the solution was removed. This washing operation was repeated three times. For destaining, a 50% acetonitrile/100 mM Tris buffer (pH: 8.0) solution was added and incubated at 37° C. for 10 minutes, and then the solution was removed. Acetonitrile was added to dehydration tubes and incubated at 37° C. for 10 minutes.

After the solution was removed, a trypsin solution was added to each tube and incubated at 37° C. overnight. The obtained solution was quenched with a trifluoroacetic acid (TFA) aqueous solution (final concentration: 0.1% v/v) and desalted using C18 Pipette Tips (Nikkyo Technos, Co., Ltd., NTCR-KT200-C18-2). After desalting, the solvent was removed by a centrifugal evaporator (Tokyo Rikakikai Co., Ltd.).

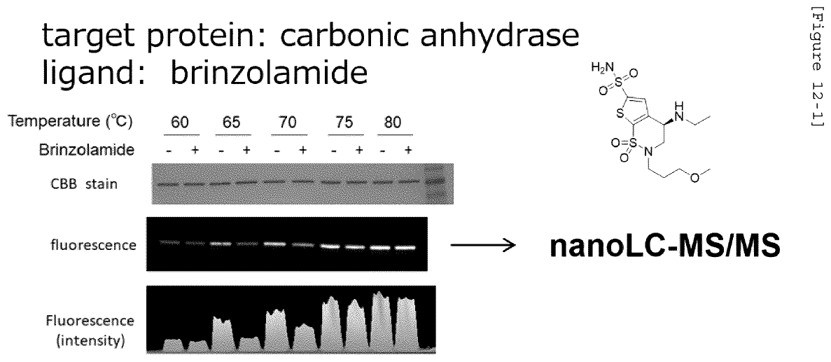

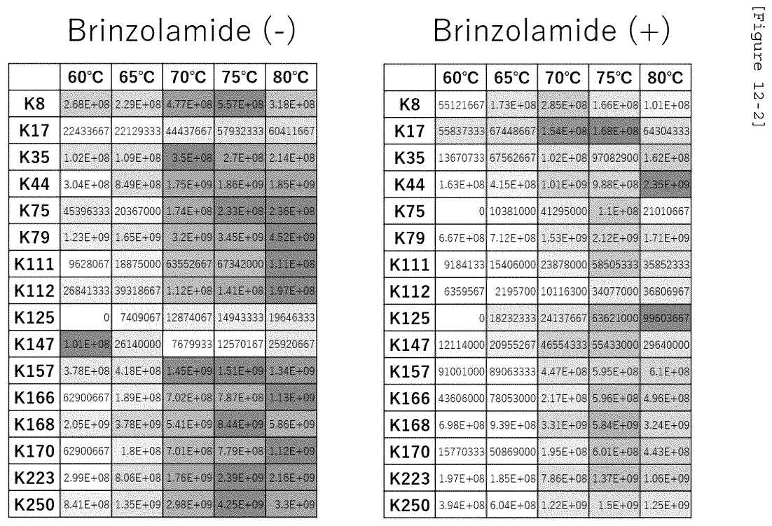

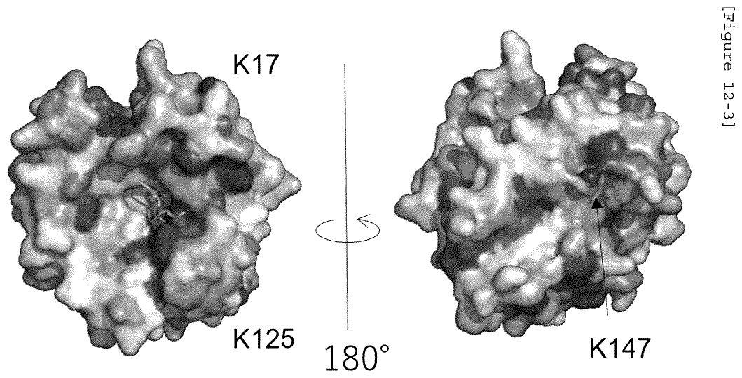

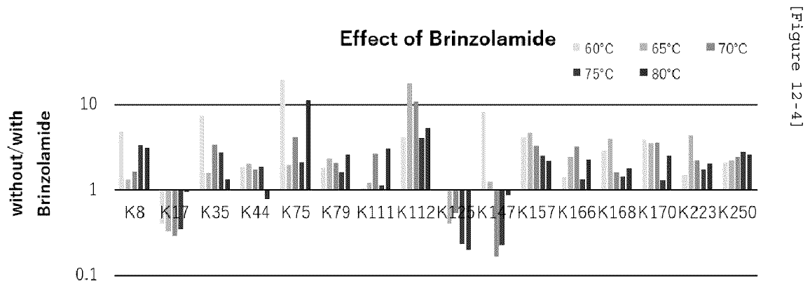

The fluorescence intensity of each trypsin-digested fragment was measured, and the degree of denaturation around Lys residues in bovine carbonic anhydrase was visualized. Further, the fluorescence intensity was compared between when heating in the presence of brinzolamide and when heating in the absence of brinzolamide, and the influence of brinzolamide on the degree of denaturation around each Lys residue was examined (FIG. 12). As shown in FIG. 12, although overall denaturation was less likely to occur due to brinzolamide, some areas were more susceptible to denaturation than others.

### [Example 14] Purification of modified fragments using anti-NBD antibody and preparation of mass spectrometry measurement samples

The band corresponding to the labelled bovine carbonic anhydrase was separated by SDS-PAGE, and the cut-out band was cut (about 1-mm pieces). The gel pieces were transferred to microtubes, and 1 mL of water was added and allowed to stand at 37° C. for 10 minutes. The solution was removed, and the washing operation was repeated three times. For destaining, 50% acetonitrile in 100 mM Tris buffer (pH: 8.0) was added and allowed to stand at 37° C. for 10 minutes, and then the solution was removed. Acetonitrile was added to dehydration tubes and allowed to stand at 37° C. for 10 minutes. After the solution was removed, a trypsin solution was added to each tube and allowed to stand at 37° C. overnight. A 10% trifluoroacetic acid (TFA) aqueous solution (final concentration: 0.4%) was added to quench the reaction. 50% acetonitrile/0.1% TFA was added and allowed to stand at 37° C. for 10 minutes. After stirring gently, centrifugation was performed, the extract was collected, and 80% acetonitrile/0.1% TFA was added and allowed to stand at 37° C. for 2 minutes. After stirring gently, centrifugation was performed, and the extract was collected. The collected extracts were concentrated together in a concentration centrifuge. Protein G Dynabeads (Invitrogen, 10003D) were added to the sample from which the organic solvent was removed, and set on a magnetic bead-collecting stand, and the supernatant was removed. After washing with Pi-Na buffer (20 mM phosphate buffer (pH: 6.8-70), 150 mM NaCl) and addition of TG buffer (20 mM Tris-HCl, pH: 8.0, 0.01% decyl 3-D-glucopyranoside (TCI, D5680)), anti-NBD antibody (Bio-Rad, 0100-0023) was added, followed by mixing by inverting at 4° C. The supernatant was removed, and after washing with Pi-Na buffer, TG buffer was added, followed by mixing by inverting with a rotator at 4° C. overnight.

The supernatant was collected from the sample tubes, washed three times with TG buffer, and then washed three times with 0.01% DG buffer (a 10-fold dilution of 0.1% decyl $-D-glucopyranoside), followed by addition of 0.15% TFA and mixing by inverting with a rotator for 10 minutes. This operation was carried out three times.

The sample was concentrated by a concentration centrifuge, and desalting was performed by using Spin-down C18 Tips (Nikkyo Technos, Co., Ltd., NTCR-KT200-C18-2). After desalting, the solvent was removed by centrifugation. 5% Acetonitrile/0.1% TFA was redissolved in the sample to prepare an LC-MS/MS sample.

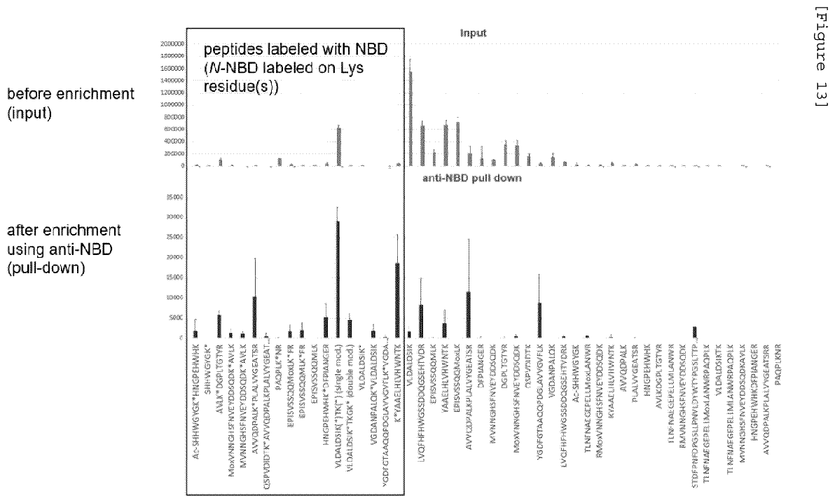

Mass spectrometry was performed as follows. Using an LC-nano-ESI-MS comprising a quadrupole time-of-flight mass spectrometer (Triple TOF (registered trademark) 5600 system; SCIEX) equipped with a nanospray ion source and a nanoLC system (Eksigent NanoLC Ultra 1D Plus; SCIEX, Massachusetts, USA), quantitative analysis of peptide fragments and protein identification were performed. The trap column used for nanoLC was NanoLC Trap ChromXP C18, 3 m 120 Å (SCIEX), and the separation column used was 12.5 cm×75 m capillary column (Nikkyo Technos, Co., Ltd.) packed with 3 m C18-silica particles. Mass spectrometry was performed while peptides were separated in the following mobile phase by the micropump (flow rate: 300 nL/min) gradient method. A: 2% acetonitrile, 0.1% formic acid, mobile phase B: acetonitrile, 0.1% formic acid aq. 0-20 minutes: 5-45% B, 20-21 minutes: 45-100% B, 21-26 minutes: 100% B. NanoLC-MS/MS data were acquired in the information-dependent acquisition mode controlled by Analyst (registered trademark) TF 1.5.1 software (SCIEX). The settings were as follows: accumulation time of 0.25 seconds, full MS (MS1, TOF-MS) scan range of 400-1250 m/z, no repetitive measurement of former target ions for 12 seconds, mass tolerance of 50 mDa, and selection of the top 10 signals for MS2 scanning in each full MS scan. The MS2 (product ion) scan accumulation time and range were 0.05 seconds and 100-1500 m/z, respectively. The entire experiment was conducted in triplicate. The MS/MS spectra were searched against the known amino acid sequence of each protein or all human protein structures obtained from UniProt using analysis software MaxQuant. For the modified peptide fragments of probe molecules, NBD modification to lysine residues (+C₆HN₃O₃, 163.0018 Da) was taken into consideration, and the modified peptide fragments were quantitatively compared. FIG. 13 shows the mass spectrometry results before and after concentration with the anti-NBD antibody.

### [Example 15] Visualization of denatured state of CA by two-dimensional electrophoresis and influence of presence or absence of ligand (BrNDB)

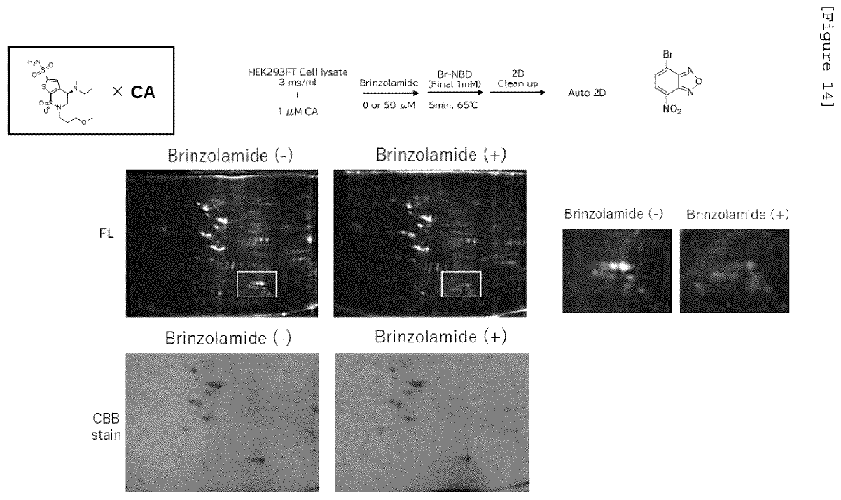

Using BrNDB as a probe, two-dimensional electrophoresis of bovine carbonic anhydrase (CA) was performed in the same manner as in Example 4 in the presence or absence of brinzolamide (FIG. 14). As shown in FIG. 14, in the presence of brinzolamide, the fluorescence intensity of CA was reduced, and it was possible to visualize the acquisition of heat resistance by the addition of brinzolamide.

### [Example 16] Visualization of denatured state of CA by two-dimensional electrophoresis and influence of presence or absence of ligand (comparison between iPrONBD and BrNBD)

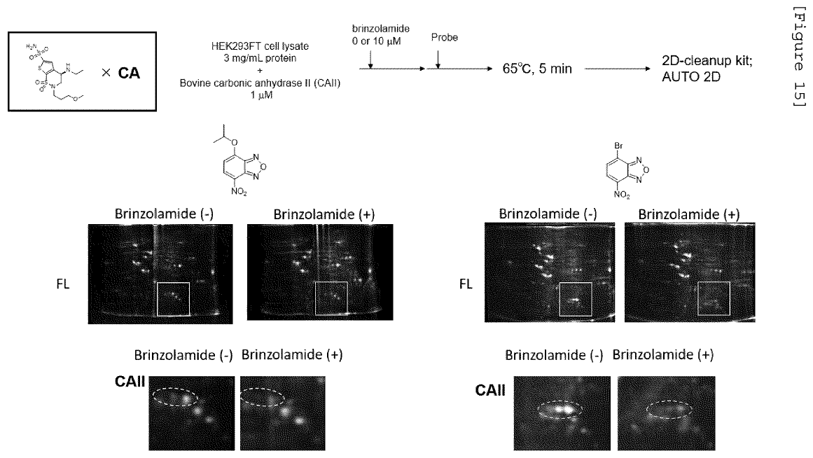

Using iPrONBD or BrNDB as a probe, two-dimensional electrophoresis was performed in the same manner as in Example 15, and the results were compared (FIG. 15). As shown in FIG. 15, the reduction in the fluorescence intensity of CA was more significant when using BrNDB. Appropriate probes vary depending on the denaturation temperature. Br-NBD is an appropriate probe under these denaturation conditions (65° C., 5 minutes)

### [Example 17] Influence of addition of geldanamycin on HSP90 precipitation and fluorescence detection of denatured HSP90 by probes

HeLa cells were scraped with a scraper and washed three times with ice-cold PBS, and a protease inhibitor cocktail (Nacalai, 25955-24) was resuspended in ice-cold M-PER buffer (Thermo Fisher Scientific, 78503) and allowed to stand on ice for 10 minutes. After centrifugation at 20,000×g for 20 minutes at 4° C., the supernatant was separated from the cell lysate. The protein concentration was determined by using the BCA method. To the supernatant (2 mg/ml), the same amount of geldanamycin (TCI, G0334; final concentration: 50 μM) and DMSO were added, followed by treatment at room temperature for 10 minutes. After a CaCl₂ buffer (0.5 μM Tris, 0.5 M NaCl, 0.2 M CaCl₂, pH: 8.0) at 10× concentration was added, the mixture was allowed to stand at room temperature for 5 minutes. A stock solution (DMSO solution) of 100 mM ONBD was added to the sample to a final concentration of 1 mM and allowed to stand at 37° C. for 30 minutes, and the solution was then transferred to a PCR tube. The PCR tube was heated using a thermal cycler (MiniAmp Plus) at each temperature for 5 minutes and then allowed to stand at room temperature. The sample was centrifuged at 20,000×g for 15 minutes at 4° C. In 1D SDS-PAGE (Bio-Rad, 4-10% acrylamide gel) of the pellet fraction, SDS-PAGE was performed while mixing with 1×SDS sample buffer and heating for 5 minutes at 95° C.

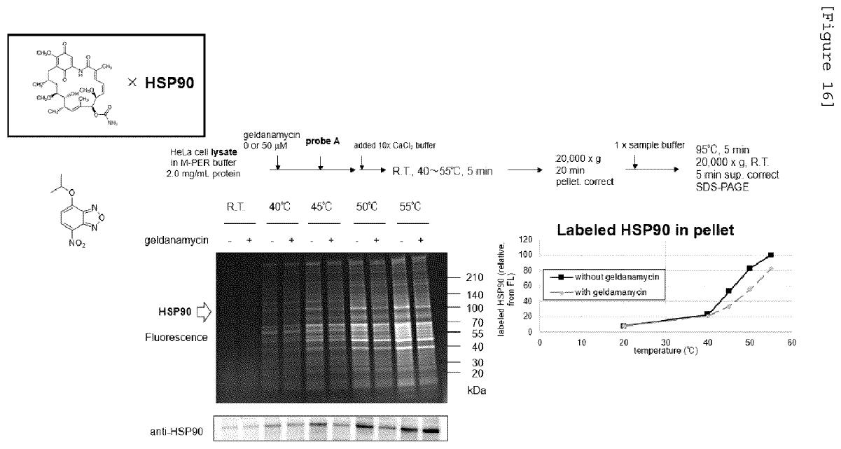

FIG. 16 shows the results of SDS-PAGE when using iPrONBD as ONBD, and relative fluorescence intensity at each heating temperature. Even when cell-intrinsic HSP90 was targeted, it was possible to visualize the acquisition of target heat denaturation resistance by ligand addition.

### [Example 18] Comparison of each probe in fluorescence detection of denatured HSP90 by probes by addition of geldanamycin

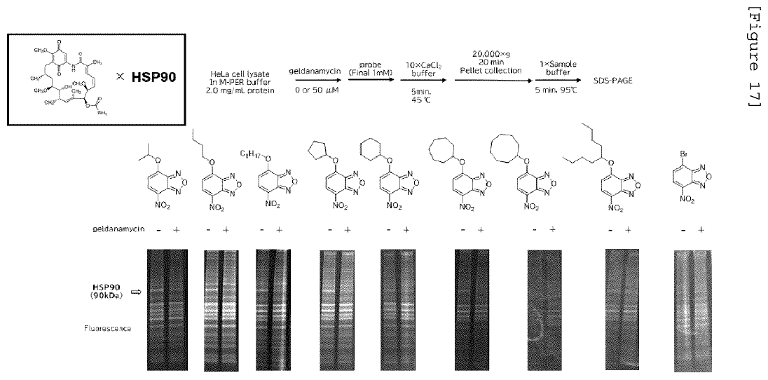

Using various probes, SDS-PAGE was performed in the same manner as in Example 17 under heating conditions of 45° C. for 5 minutes (FIG. 17). The right-side probes were suitable for detection of proteins at higher temperatures, but were not suitable for detection of proteins denatured on the low-temperature side (around 45° C.), such as HSP90. It was desirable to use the left-handed, highly reactive probes.

### [Example 19] Reaction of probe molecules in intracellular environment (comparison of denatured HSP90 by addition of geldanamycin)

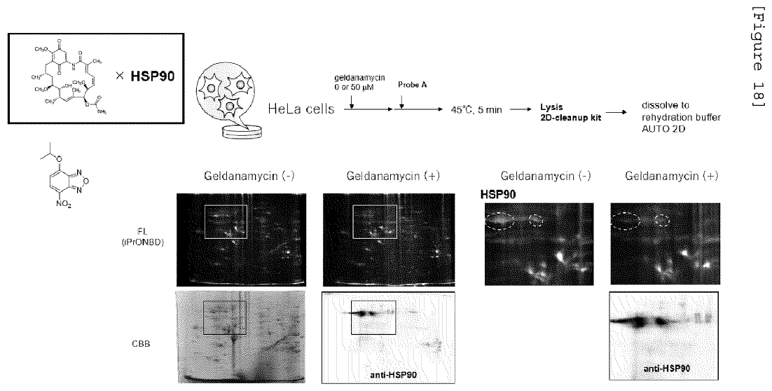

HeLa cells were scraped with a scraper and washed three times with ice-cold PBS, and a protease inhibitor cocktail (Nacalai, 25955-24) was resuspended in ice-cold M-PER buffer (Thermo Fisher Scientific, 78503) and allowed to stand on ice for 10 minutes. After centrifugation at 20,000×g for 20 minutes at 4° C., the supernatant was separated from the cell lysate. The protein concentration was determined by using the BCA method. To the supernatant (2 mg/ml), the same amount of geldanamycin (TCI, G0334; final concentration: 50 μM) and DMSO were added, followed by treatment at room temperature for 10 minutes. After a CaCl₂ buffer (0.5 M Tris, 0.5 M NaCl, 0.2 M CaCl₂, pH: 8.0) at 10× concentration was added, the mixture was allowed to stand at room temperature for 5 minutes. A stock solution (DMSO solution) of 100 mM iPrONBD was added to the sample to a final concentration of 1 mM and allowed to stand at 37° C. for 30 minutes, and the solution was then transferred to a PCR tube. The PCR tube was heated using a thermal cycler (MiniAmp Plus) at 45° C. for 5 minutes and then allowed to stand at room temperature. A sample was prepared according to the protocol of the ReadyPrep 2-D cleanup kit (Bio-Rad), and the final pellets obtained were dissolved in 15 μL of swelling buffer (containing ampolyte, pH: 3-10) included with the Auto2D Glycine Type Reagent Kit, followed by two-dimensional electrophoresis (FIG. 18). Even as a result of reacting the probe in living cells and analyzing the total protein in the cell lysate, it was possible to visualize changes in the heat denaturation resistance of the target HSP90 by ligand addition.

### [Example 20] Protein identification from spots in two-dimensional electrophoresis

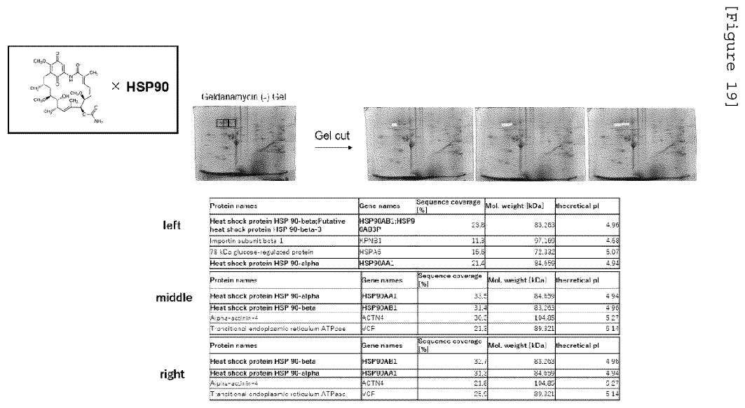

Two-dimensional electrophoresis was performed according to the long-term protocol of Auto 2D Plus (Merck), and fluorescence images of the resulting gel were obtained using a fluorescence imager (Vilver Lourmat, Fusion Solo S). Thereafter, all proteins contained in the gel were stained by CBB staining (Nacalai, CBB Stain One Super, 11642-44) according to the manufacturer's protocol, and images were taken in the same manner. The spot of the target to be identified was cut out, gel pieces were transferred to microtubes, and 1 mL of water was added and allowed to stand at 37° C. for 10 minutes. The solution was removed, and the washing operation was repeated three times. For destaining, 50% acetonitrile in 100 mM Tris buffer (pH: 8.0) was added and allowed to stand at 37° C. for 10 minutes, and then the solution was removed. Acetonitrile was added to dehydration tubes and allowed to stand at 37° C. for 10 minutes. After the solution was removed, a trypsin solution (Promega, Trypsin Gold) was added to each tube and allowed to stand at 37° C. overnight. A 10% trifluoroacetic acid (TFA) aqueous solution (final concentration: 0.4%) was added to quench the reaction. 50% acetonitrile/0.1% TFA was added and allowed to stand at 37° C. for 10 minutes. After stirring gently, centrifugation was performed, the extract was collected, and 80% acetonitrile/0.1% TFA was added and allowed to stand at 37° C. for 2 minutes. After stirring gently, centrifugation was performed, and the extract was collected. The collected extracts were dried together in a concentration centrifuge, and the sample redissolved in 5% acetonitrile/0.1% TFA was subjected to mass spectrometry to identify proteins (FIG. 19). Mass spectrometry was performed in the same manner as in Example 14.

All publications, patents, and patent applications cited in the present specification should be incorporated by reference in the present specification.

### INDUSTRIAL APPLICABILITY

The present invention can be used in industrial fields related to denatured proteins and aggregated proteins.

1. A method for visualizing the denatured state or aggregated state of a protein, the method comprising:
a step of bringing the protein into contact with a compound represented by the following general formula (I): 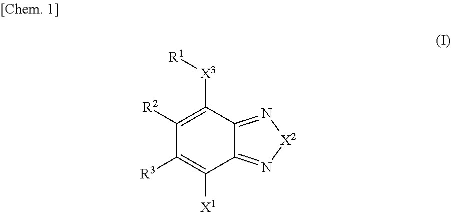
[wherein R¹ represents a C₁₋₁₂ linear alkyl group (provided that one or more —CH₂—CH₂— in the alkyl group are optionally replaced with —CO—NH—), a C₃₋₁₀ branched alkyl group (provided that one or more non-adjacent carbon atoms in the alkyl group are optionally replaced with a nitrogen atom or an oxygen atom), a C₃₋₁₀ cycloalkyl group optionally substituted with a substituent (provided that one or more non-adjacent carbon atoms in the cycloalkyl group are optionally replaced with a nitrogen atom or an oxygen atom), an aryl group optionally substituted with a substituent, a heteroaryl group optionally substituted with a substituent, a group represented by —(CH₂—CH₂—O—)ₙ—CH₃ (n represents an integer of 1 to 4), an adamantyl group, or a halogen atom; R² and R³ each independently represent a hydrogen atom or a C₁₋₃ linear or branched alkyl group; X¹ represents a nitro group or a group represented by —SO₂NR⁴R⁵(wherein R⁴ and R⁵ each independently represent a C₁₋₃ linear or branched alkyl group); X² represents a group represented by any of the following formulas (i) to (v): 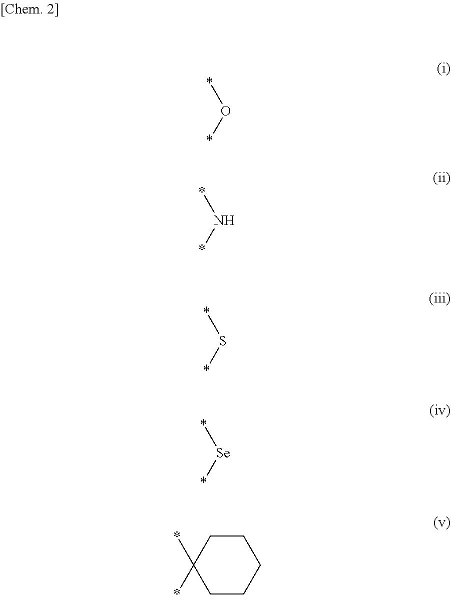
(wherein * represents a bond and binds to a nitrogen atom in the general formula (I)); and
X³ does not exist or represents an oxygen atom or a sulfur atom]; and
a step of detecting fluorescence of a reaction product of the compound represented by the general formula (I).

2. The method according to claim 1, wherein R¹ in the general formula (I) is a C₄₋₁₂ linear alkyl group (provided that one or more —CH₂—CH₂— in the alkyl group are optionally replaced with —CO—NH—), a C₃₋₅ branched alkyl group (provided that one or more non-adjacent carbon atoms in the alkyl group are optionally replaced with a nitrogen atom or an oxygen atom), a C₄₋₆ cycloalkyl group optionally substituted with a substituent (provided that one or more non-adjacent carbon atoms in the cycloalkyl group are optionally replaced with a nitrogen atom or an oxygen atom), a phenyl group optionally substituted with a substituent, a 2-thienyl group optionally substituted with a substituent, a 3-thienyl group optionally substituted with a substituent, a 2-furanyl group optionally substituted with a substituent, a 3-furanyl group optionally substituted with a substituent, a 2-pyridyl group optionally substituted with a substituent, a 3-pyridyl group optionally substituted with a substituent, a 4-pyridyl group optionally substituted with a substituent, or a group represented by —(CH₂—CH₂—O—)ₙ—CH₃ (n represents 2 or 3).

3. The method according to claim 1, wherein R¹ in the general formula (I) is an n-butyl group, an n-octyl group, an iso-propyl group, a sec-butyl group, a tert-butyl group, a 3-pentyl group, a 1-methyl-2-methylamino-ethyl group, a cycloalkyl group, a 3-azetidyl group, a 3-piperidinyl group, a 3-aminomethylcyclobutyl group, a 3-aminocyclopentyl group, a 4-aminocyclohexyl group, a phenyl group, a 2-thienyl group, a 3-thienyl group, a 2-furanyl group, a 3-furanyl group, a 2-pyridyl group, a 3-pyridyl group, a 4-pyridyl group, or a group represented by —(CH₂—CH₂—O—)₂—CH₃.

4. The method according to claim 1, wherein R¹ in the general formula (I) is an n-butyl group, an n-octyl group, an iso-propyl group, a 3-pentyl group, or a group represented by —(CH₂—CH₂—O—)₂—CH₃.

5. A visualizing agent for the denatured state or aggregated state of a protein, the visualizing agent comprising a compound represented by the following general formula (I): 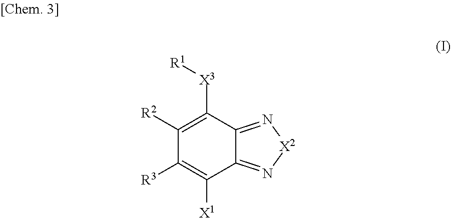
[wherein R¹ represents a C₁₋₁₂ linear alkyl group (provided that one or more —CH₂—CH₂— in the alkyl group are optionally replaced with —CO—NH—), a C₃₋₁₀ branched alkyl group (provided that one or more non-adjacent carbon atoms in the alkyl group are optionally replaced with a nitrogen atom or an oxygen atom), a C₃₋₁₀ cycloalkyl group optionally substituted with a substituent (provided that one or more non-adjacent carbon atoms in the cycloalkyl group are optionally replaced with a nitrogen atom or an oxygen atom), an aryl group optionally substituted with a substituent, a heteroaryl group optionally substituted with a substituent, a group represented by —(CH₂—CH₂—O—)ₙ—CH₃ (n represents an integer of 1 to 4), an adamantyl group, or a halogen atom; R² and R³ each independently represent a hydrogen atom or a C₁₋₃ linear or branched alkyl group; X¹ represents a nitro group or a group represented by —SO₂NR⁴R⁵(wherein R⁴ and R⁵ each independently represent a C₁₋₃ linear or branched alkyl group); X² represents a group represented by any of the following formulas (i) to (v): 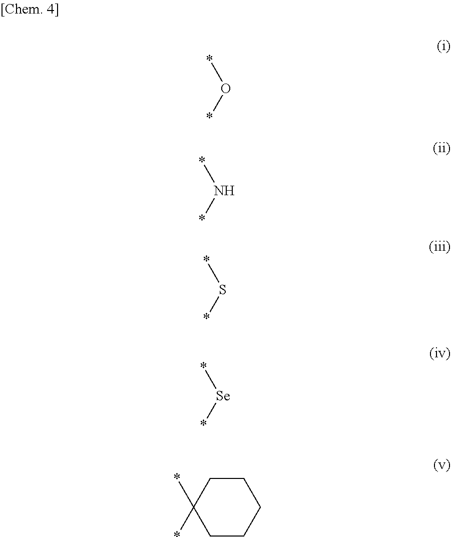
(wherein * represents a bond and binds to a nitrogen atom in the general formula (I)); and
X³ does not exist or represents an oxygen atom or a sulfur atom].

6. The visualizing agent according to claim 5, wherein R¹ in the general formula (I) is a C₄₋₁₂ linear alkyl group (provided that one or more —CH₂—CH₂— in the alkyl group are optionally replaced with —CO—NH—), a C₃₋₅ branched alkyl group (provided that one or more non-adjacent carbon atoms in the alkyl group are optionally replaced with a nitrogen atom or an oxygen atom), a C₄₋₆ cycloalkyl group optionally substituted with a substituent (provided that one or more non-adjacent carbon atoms in the cycloalkyl group are optionally replaced with a nitrogen atom or an oxygen atom), a phenyl group optionally substituted with a substituent, a 2-thienyl group optionally substituted with a substituent, a 3-thienyl group optionally substituted with a substituent, a 2-furanyl group optionally substituted with a substituent, a 3-furanyl group optionally substituted with a substituent, a 2-pyridyl group optionally substituted with a substituent, a 3-pyridyl group optionally substituted with a substituent, a 4-pyridyl group optionally substituted with a substituent, or a group represented by —(CH₂—CH₂—O—)ₙ—CH₃ (n represents 2 or 3).

7. The visualizing agent according to claim 5, wherein R¹ in the general formula (I) is an n-butyl group, an n-octyl group, an iso-propyl group, a sec-butyl group, a tert-butyl group, a 3-pentyl group, a 1-methyl-2-methylamino-ethyl group, a cycloalkyl group, a 3-azetidyl group, a 3-piperidinyl group, a 3-aminomethylcyclobutyl group, a 3-aminocyclopentyl group, a 4-aminocyclohexyl group, a phenyl group, a 2-thienyl group, a 3-thienyl group, a 2-furanyl group, a 3-furanyl group, a 2-pyridyl group, a 3-pyridyl group, a 4-pyridyl group, or a group represented by —(CH₂—CH₂—O—)₂—CH₃.

8. The visualizing agent according to claim 5, wherein R¹ in the general formula (I) is an n-butyl group, an n-octyl group, an iso-propyl group, a 3-pentyl group, or a group represented by —(CH₂—CH₂—O—)₂—CH₃.

9. A method for specifying a denatured protein or an aggregated protein in a sample, the method comprising:
a step of bringing the sample into contact with a compound represented by the following general formula (I): 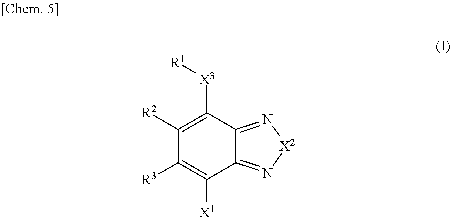
[wherein R¹ represents a C₁₋₁₂ linear alkyl group (provided that one or more —CH₂—CH₂— in the alkyl group are optionally replaced with —CO—NH—), a C₃₋₁₀ branched alkyl group (provided that one or more non-adjacent carbon atoms in the alkyl group are optionally replaced with a nitrogen atom or an oxygen atom), a C₃₋₁₀ cycloalkyl group optionally substituted with a substituent (provided that one or more non-adjacent carbon atoms in the cycloalkyl group are optionally replaced with a nitrogen atom or an oxygen atom), an aryl group optionally substituted with a substituent, a heteroaryl group optionally substituted with a substituent, a group represented by —(CH₂—CH₂—O—)ₙ—CH₃ (n represents an integer of 1 to 4), an adamantyl group, or a halogen atom; R² and R³ each independently represent a hydrogen atom or a C₁₋₃ linear or branched alkyl group; X¹ represents a nitro group or a group represented by —SO₂NR⁴R⁵(wherein R⁴ and R⁵ each independently represent a C₁₋₃ linear or branched alkyl group); X² represents a group represented by any of the following formulas (i) to (v): 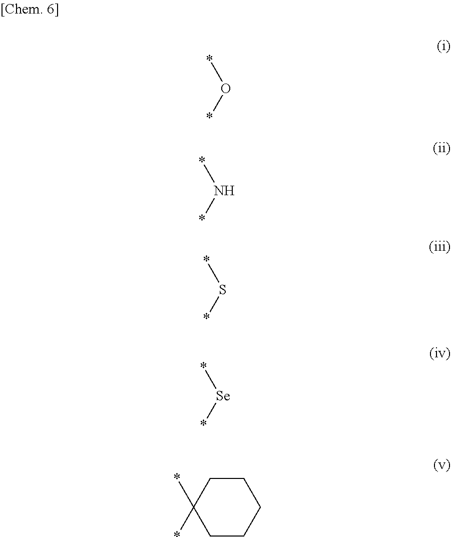
(wherein * represents a bond and binds to a nitrogen atom in the general formula (I)); and
X³ does not exist or represents an oxygen atom or a sulfur atom];
a step of separating proteins in the sample; and
a step of detecting fluorescence of a reaction product of the compound represented by the general formula (I).

10. The method according to claim 9, wherein R¹ in the general formula (I) is a C₄₋₁₀ linear alkyl group (provided that one or more —CH₂—CH₂— in the alkyl group are optionally replaced with —CO—NH—), a C₃₋₅ branched alkyl group (provided that one or more non-adjacent carbon atoms in the alkyl group are optionally replaced with a nitrogen atom or an oxygen atom), a C₄₋₆ cycloalkyl group optionally substituted with a substituent (provided that one or more non-adjacent carbon atoms in the cycloalkyl group are optionally replaced with a nitrogen atom or an oxygen atom), a phenyl group optionally substituted with a substituent, a 2-thienyl group optionally substituted with a substituent, a 3-thienyl group optionally substituted with a substituent, a 2-furanyl group optionally substituted with a substituent, a 3-furanyl group optionally substituted with a substituent, a 2-pyridyl group optionally substituted with a substituent, a 3-pyridyl group optionally substituted with a substituent, a 4-pyridyl group optionally substituted with a substituent, or a group represented by —(CH₂—CH₂—O—)ₙ—CH₃ (n represents 2 or 3).

11. The method according to claim 9, wherein R¹ in the general formula (I) is an n-butyl group, an n-octyl group, an iso-propyl group, a sec-butyl group, a tert-butyl group, a 3-pentyl group, a 1-methyl-2-methylamino-ethyl group, a cycloalkyl group, a 3-azetidyl group, a 3-piperidinyl group, a 3-aminomethylcyclobutyl group, a 3-aminocyclopentyl group, a 4-aminocyclohexyl group, a phenyl group, a 2-thienyl group, a 3-thienyl group, a 2-furanyl group, a 3-furanyl group, a 2-pyridyl group, a 3-pyridyl group, a 4-pyridyl group, or a group represented by —(CH₂—CH₂—O—)₂—CH₃.

12. The method according to claim 9, wherein R¹ in the general formula (I) is an n-butyl group, an n-octyl group, an iso-propyl group, a 3-pentyl group, or a group represented by —(CH₂—CH₂—O—)₂—CH₃.

13. A method for identifying a protein binding to a test compound, the method comprising the following steps:
(1) adding the test compound and a fluorescence substance to a protein-containing sample;
(2) heating the sample of step (1);
(3) separating proteins contained in the sample of step (2);
(4) measuring fluorescence intensity of each protein separated in step (3);
(5) adding a fluorescence substance to a protein-containing sample;
(6) heating the sample of step (5);
(7) separating proteins contained in the sample of step (6);
(8) measuring fluorescence intensity of each protein separated in step (7); and
(9) comparing the fluorescence intensity of each protein measured in step (4) and the fluorescence intensity of each protein measured in step (8), and identifying a protein in which the fluorescence intensity measured in step (4) is lower than the fluorescence intensity measured in step (8) as a protein binding to the test compound,
wherein the fluorescence substance is a compound represented by the following general formula (I): 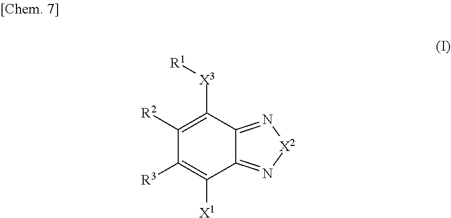
[wherein R¹ represents a C₁₋₁₂ linear alkyl group (provided that one or more —CH₂—CH₂— in the alkyl group are optionally replaced with —CO—NH—), a C₃₋₁₀ branched alkyl group (provided that one or more non-adjacent carbon atoms in the alkyl group are optionally replaced with a nitrogen atom or an oxygen atom), a C₃₋₁₀ cycloalkyl group optionally substituted with a substituent (provided that one or more non-adjacent carbon atoms in the cycloalkyl group are optionally replaced with a nitrogen atom or an oxygen atom), an aryl group optionally substituted with a substituent, a heteroaryl group optionally substituted with a substituent, a group represented by —(CH₂—CH₂—O—)ₙ—CH₃ (n represents an integer of 1 to 4), an adamantyl group, or a halogen atom; R² and R³ each independently represent a hydrogen atom or a C₁₋₃ linear or branched alkyl group; X¹ represents a nitro group or a group represented by —SO₂NR⁴R⁵(wherein R⁴ and R⁵ each independently represent a C₁₋₃ linear or branched alkyl group); X² represents a group represented by any of the following formulas (i) to (v): 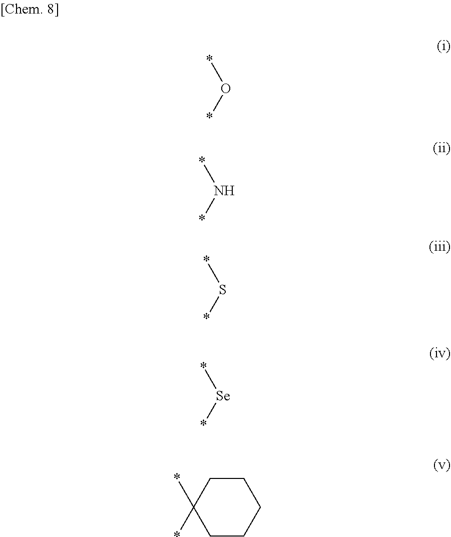
(wherein * represents a bond and binds to a nitrogen atom in the general formula (I)); and
X³ does not exist or represents an oxygen atom or a sulfur atom].

14. The method according to claim 13, wherein R¹ in the general formula (I) is a C₄₋₁₂ linear alkyl group (provided that one or more —CH₂—CH₂— in the alkyl group are optionally replaced with —CO—NH—), a C₃₋₅ branched alkyl group (provided that one or more non-adjacent carbon atoms in the alkyl group are optionally replaced with a nitrogen atom or an oxygen atom), a C₄₋₆ cycloalkyl group optionally substituted with a substituent (provided that one or more non-adjacent carbon atoms in the cycloalkyl group are optionally replaced with a nitrogen atom or an oxygen atom), a phenyl group optionally substituted with a substituent, a 2-thienyl group optionally substituted with a substituent, a 3-thienyl group optionally substituted with a substituent, a 2-furanyl group optionally substituted with a substituent, a 3-furanyl group optionally substituted with a substituent, a 2-pyridyl group optionally substituted with a substituent, a 3-pyridyl group optionally substituted with a substituent, a 4-pyridyl group optionally substituted with a substituent, or a group represented by —(CH₂—CH₂—O—)ₙ—CH₃ (n represents 2 or 3).

15. The method according to claim 13, wherein R¹ in the general formula (I) is an n-butyl group, an n-octyl group, an iso-propyl group, a sec-butyl group, a tert-butyl group, a 3-pentyl group, a 1-methyl-2-methylamino-ethyl group, a cycloalkyl group, a 3-azetidyl group, a 3-piperidinyl group, a 3-aminomethylcyclobutyl group, a 3-aminocyclopentyl group, a 4-aminocyclohexyl group, a phenyl group, a 2-thienyl group, a 3-thienyl group, a 2-furanyl group, a 3-furanyl group, a 2-pyridyl group, a 3-pyridyl group, a 4-pyridyl group, or a group represented by —(CH₂—CH₂—O—)₂—CH₃.

16. The method according to claim 13, wherein R¹ in the general formula (I) is an n-butyl group, an n-octyl group, an iso-propyl group, a 3-pentyl group, or a group represented by —(CH₂—CH₂—O—)₂—CH₃.

17. A compound represented by the following general formula (Ia), (Ib), or (Ic): 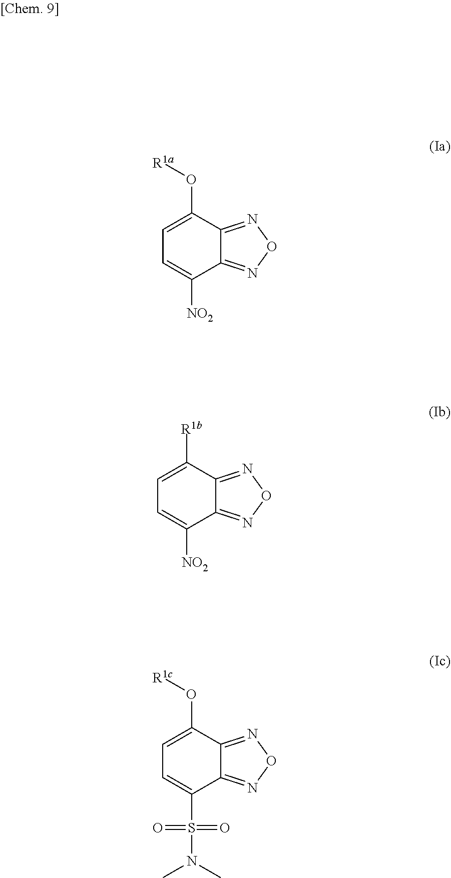
[wherein R^{1a} represents an iso-propyl group, an n-butyl group, a 3-pentyl group, a cyclobutyl group, a cyclopentyl group, a cyclohexyl group, a cycloheptyl group, a cyclooctyl group, a 2-adamantyl group, an n-hexyl group, an n-octyl group, an n-decyl group, an n-dodecyl group, an iso-butyl group, a 5-nonyl group, or a diethylene glycol group; R^{1b} represents a bromine atom or an iodine atom; and R^{1c} represents a methyl group or an iso-propyl group].
